(19) United States
(12) Patent Application Publication
(10) Pub. No.: US 2021/0223269 A1
(43) Pub. Date: Jul. 22, 2021

(54) BIOMARKERS FOR DETECTION OF CORONARY ARTERY DISEASE AND ITS MANAGEMENT

(71) Applicants: Oregon State University, Corvallis, OR (US); Oregon Health & Science University, Portland, OR (US)

(72) Inventors: Manuel Garcia-Jaramillo Rodriguez, Corvallis, OR (US); Gerd Bobe, Corvallis, OR (US); Claudia Susanne Maier, Corvallis, OR (US); Donald Bradley Jump, Philomath, OR (US); Armando Alcázar-Magaña, Apatzingan (MX); Dai-Trang Elizabeth Le, Portland, OR (US); Sanjiv Kaul, Portland, OR (US); Nabil J. Alkayed, West Linn, OR (US)

(73) Assignees: Oregon State University, Corvallis, OR (US); Oregon Health & Science University, Portland, OR (US)

(21) Appl. No.: 17/156,431

(22) Filed: Jan. 22, 2021

Related U.S. Application Data
(60) Provisional application No. 62/964,510, filed on Jan. 22, 2020.

(51) Int. Cl. G01N 33/92

(52) U.S. Cl. CPC G01N 33/92

(57) ABSTRACT

The present disclosure provides biomarkers for the detection of coronary artery disease. The biomarkers comprise oxylipins and the detection, identification, and quantification can provide a means to diagnose, prognose, and manage subject at risk for cardiovascular disease. Methods of also provided for the detection and quantification of the oxylipins, for treating, and for predicting the survival of a subject at high risk for coronary artery disease.

### CROSS-REFERENCE TO RELATED APPLICATION

This application claims the benefit of U.S. Provisional Application No. 62/964,510, filed Jan. 22, 2020, the disclosure of which is incorporated herein in its entirety.

### STATEMENT OF GOVERNMENT LICENSE RIGHTS

This invention was made with government support under contract DK112360 (DBJ) awarded by the National Institutes of Health. The government has certain rights in the invention.

### BACKGROUND

Coronary Artery Disease (CAD) is the most common type of heart disease worldwide, where it is the leading cause of death and predicted to remain so for at least the next two decades (Roth et al., 2017; Papidipati et al., 2013; Nayeem, 2018). Each year, approximately 3.8 million men and 3.4 million women die from CAD (WHO, 2004). It is estimated that in 2020 it will be responsible for a total of 11 million deaths globally (Mathers et al., 2006). In the U.S. alone, someone suffers a coronary event every 26 seconds, and someone dies from one every minute. According to the American Heart Association, 770,000 Americans suffered a new acute coronary syndrome in 2008, and a further 430,000 experienced a recurrent event. An additional 190,000 silent first heart attacks occur each year (Mozzafarian et al., 2016).

Traditional CAD risk factors such as diabetes, smoking, hypertension, hyperlipidemia, and family history of premature cardiovascular disease (CVD), as well as non-traditional risk factors of rheumatic inflammatory disease, human immunodeficiency disease, and gestational diabetes can assist clinicians in decisions for CAD primary prevention but have limited efficacy in high CAD risk patient management (Hajar, 2017). This is currently done using expensive invasive tests, such as exercise stress testing (without or with concomitant imaging for myocardial perfusion and/or function) or measuring the coronary calcium score on x-ray computed tomography (CT). Our objective is to develop a point-of-care blood test that could assist in decision making regarding CAD patient management.

Ruptured arterial plaques are a major reason for adverse cardiac events with resultant thrombus formation that partially or completely impairs blood flow to the heart (Ambrose and Singh, 2015. The progression from asymptomatic to ruptured arterial plaques involves lipid oxidation and inflammation (Bentzon et al., 2014; Rafieian-Kopaei et al., 2014). Conventional indicators of lipid oxidation include secondary products such as 4-hydroxynonenal (4-HNE)(Zhong and Yin, 2014), malondialdehyde (MDA)(Gawel et al., 2004), and oxidized low-density lipoproteins (Ox-LDL)(Parthasarathy et al., 2010), and its associated oxidized-phospholipids (Catala, 2009). Technological advancements in soft ionization tandem mass spectrometry (MS/MS) have allowed multiplexed quantification of oxidized lipids in one analytical run and with it the emergence of isoprostanes and oxylipins as indicators of oxidative tissue injuries, implicating oxidative tissue injuries in the pathology of a variety of chronic diseases (Nayeem, 2018; Tourdot et al., 2014).

Some inflammatory biomarkers are associated with CAD and provide prognostic information. They have, however, not been shown to have a diagnostic role. For instance, patients with high levels of C-reactive protein (CRP) are at increased risk for coronary events and diabetes (Kuller et al., 1996). A large-scale prospective study documented a strong association between the predictive power of CRP and CAD risk, with CRP levels being a more reliable biomarker of cardiovascular disease than LDL-cholesterol (Ridker et al., 2004). Similarly, elevated levels of Serum Amyloid A (SAA) have been reported to correlate with severity of CAD and increased risk of complications. SAA has been used to predict mortality in CAD patients (Johnson et al., 2004; Harb et al., 2002). Likewise, myeloperoxidase (MPO) levels are elevated in CAD and correlate with its extent (Liu et al., 2012).

Oxylipins, oxidized long and very long chain polyunsaturated fatty acids (PUFA), which are derived from phospholipids. Oxylipins can be classified based on their fatty acid (FA) precursor (FIGS. 1A and 1B) or their synthesis pathways and can induce pro-inflammatory or anti-inflammatory pathways. The dominant precursors of oxylipins are the more pro-inflammatory omega-6 PUFA linoleic acid (LA; C18:2 n−6) and arachidonic acid (ARA; C20:4; n−6), and the more anti-inflammatory precursors omega-3 PUFA linolenic acid (LNA; C18:3 n−3), eicosapentaenoic acid (EPA; C20:5 n−3) and docosahexaenoic acid (DHA; C22:6 n−3). The dominant oxylipin biosynthesis pathways are named after the enzymes involved in their production: lipoxygenases (LOX), cyclooxygenases (COX), as well as cytochrome P450 (CYP450) epoxygenases and hydroxylases, and soluble epoxide hydrolases. Reactive oxygen species (ROS) can also initiate the formation of a few specific oxylipins from PUFAs (Gabbs et al., 2015). FIGS. 1A and 1B illustrate the metabolic pathways of the Oxylipins relevant to this disclosure and the findings disclosed herein. Table 2 lists the nomenclature of these molecules, the enzymes and the substrates.

Oxylipins have been extensively studied in animal models but less so in humans. Prior human studies with limited sample sizes reported elevated concentrations of ARA-derived oxylipins in unstable arterial plaques (Mallat et al., 1999) and ischemic heart tissue (Lundqvist et al., 2016). Elevated circulating concentrations were observed in individuals after cardiac surgery (Strassburg et al., 2012) and those experiencing adverse cardiac events on follow up (Zu et al., 2016; Caligiuri et al., 2017). Furthermore, CAD patients had higher circulating concentrations of ARA-derived oxylipins than non-CAD adults (Shishehbor et al., 2006; Xu et al., 2013; Auguet et al., 2018).

There are currently no reliable biomarkers for the presence, extent or severity of CAD. Traditional risk factors are poor predictors of CAD, necessitating exercise stress testing (without or with concomitant imaging for myocardial perfusion and/or function) or, more recently, measuring the coronary artery calcium score (CAC) on x-ray computed tomography. The severity of CAD is confirmed by invasive coronary angiography prior to any intervention. All these tests are expensive and, for most people, inaccessible. Hence, a simple, inexpensive blood test indicating the presence or absence as well as severity of CAD would be very valuable.

This disclosure provides methods useful for detecting and/or diagnosing the presence, extent and/or severity of CAD. For example, methods for detecting and/or diagnosing the presence, extent and/or severity of CAD, can include one or more of the following steps: identifying a suitable subject, obtaining a biofluid sample, for example, a blood sample from the subject, analyzing the sample in vitro to determine the presence or amount of at least one oxylipin or ratio of oxylipins, and using the presence or amount and/or at least one oxylipin or a ratio of oxylipins to determine the CAD status of the subject. For example, an adult subject with obstructed coronary arteries with ≥70% stenosis, plasma oxylipin panels can diagnose the number of obstructed coronary arteries and can predict median 5-year outcomes. Optionally, the CAD status of the subject is used to determine proper testing, monitoring, and/or a therapeutic approach to the subject's CAD.

Biomarkers of the present disclosure can be obtained from a subject in a variety of ways that are standard in the art. Although suitable subjects included any person, it is preferred that the subject is a person with suspected coronary disease. By way of example, as subject may be suspected of having CAD if the subject suffers from symptoms associated with CAD, such as chest pain or shortness of breath during exertion, or if the subject has greater than one risk factor for CAD, such as family history of premature coronary disease, smoking, high blood pressure, high cholesterol, diabetes, chronic kidney disease. Suitable subjects may also be determined based on age or other acceptable screening criteria. For example, a suitable subject may be age fifty or older.

The biomarkers of the present disclosure can be measured and analyzed by means that are standard in the art. For example, biomarkers can be measured using mass spectrometry, immunoassays (such as ELISA), aptamers, or other in vitro means and methods standard now or in the further for detection of biomarkers.

Although most of the methods used in detecting and/or quantitating the presence of among of a oxylipin in a subject or a group of subjects may be well known in the art, the results obtained and disclosed herein provide a vastly improved method for detecting and/or diagnosing CAD such that the treatment of a subject can be selected to improve the clinical outcome for the subject. The methods provided are significantly less expensive and easier to perform making the detection and/or diagnosis of CAD available to a significantly larger number of subjects.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

The present disclosure provides an in vitro method for identifying a modified concentration (level) of at least one oxylipin in a biofluid sample obtained from a subject with a risk of coronary artery disease (CAD), the method comprising the steps of: a. obtaining a biofluid sample from the subject; b. detecting the concentration or level of at least two oxylipins, and c. comparing the concentration (level) of the at least two oxylipins in the biofluid sample from the subject with a risk of CAD to a control level of the at least two oxylipins in at least one reference standard; wherein the concentration difference for each of the at least two oxylipins is decreased with the increase in the number of disease arteries, or at least two are decreased and one is increased wherein the subject has a higher chance of survival.

In certain embodiments disclosed herein the method comprises the detection of the level of the at least two oxylipins that are oxygenated omega-6 PUFA LA and ARA. In certain embodiments described herein the method comprises the detection of the concentration of level of at least two oxylipins that are LA-derived mid-chain HODE and/or ARA-derived mid-chain HETE.

In certain more specific embodiments of the method disclosed here the method comprises the detection of the at least two oxylipins comprising Leukotriene B4, 9(S)-HODE, 13(S)-HODE, 16(17)-DiHDPA, 13(14)-DiHDPA, 19(20)-EPDPA, 19(20)-DiHDPA, 10(11)-DiHDPA, 10(11)-EpDPA, or 7(8)-DiHDPA, or combinations thereof. In a more specific embodiment the method comprises the detection of the level or concentration of at least two oxylipins comprising Leukotriene B4, 19(20)-DiHDPA, 13(14)-DiHDPA, and DiHDPA. In yet another more specific embodiment presented herein comprises detection of the concentration and/or level of at least two oxylipins comprising 9(S)-HODE, 16(17)-DiHDPA, 19(20)-EPDPA, 19(20)-DiHDPA, and 7(8)-DiHDPA. In still yet another more particular embodiment the method comprises the detection of at least oxylipins comprising 13(S)-HODE, and 10(11)-EpDPA.

The method as described herein can be carried out on a sample from a subject in need of testing, wherein the sample comprises a biofluid sample. The biofluid sample can comprise, for example, but not limitation, a blood sample, a serum sample, a plasma sample, a urine sample, or a cerebrospinal fluid sample, and the like.

In certain embodiments of the method the oxylipins can be detected by mass spectrometry (MS), nuclear magnetic resonance (NMR) spectroscopy, HPLC-UV, infrared spectroscopy, a biochemical assay, or an immunoassay in the biofluid sample.

The present disclosure also provides a method for treating CAD in a subject, wherein the method comprises: a. obtaining the results of an in vitro method, wherein said method comprises: (i.) obtaining a biofluid sample from the subject; (ii.) detecting the concentration or level of at least two oxylipins; and (iii.) comparing the concentration or level of the at least two oxylipins in the biofluid sample from the subject with a risk of CAD to a control level of the at least two oxylipins in at least one reference standard from a subject not at risk of CAD; wherein the concentration difference or level for each of the at least two oxylipins is decreased with the increase in the number of disease arteries, and (b.) treating the subject with coronary stent placement, or coronary artery bypass graft (CABG) surgery.

In a certain embodiment of the method of treatment disclosed herein the at least two oxylipins are oxygenated omega-6 PUFA LA and ARA. In one embodiment of the method of treatment disclosed herein the at least two oxylipins are LA-derived mid-chain HODE and/or ARA-derived mid-chain HETE.

In another embodiment of the method of treatment the at least two oxylipins comprise Leukotriene B4, 9(S)-HODE, 13(S)-HODE, 16(17)-DiHDPA, 13(14)-DiHDPA, 19(20)-EPDPA, 19(20)-DiHDPA, 10(11)-DiHDPA, 10(11)-EpDPA, or 7(8)-DiHDPA, or combinations thereof.

In yet another embodiment of the method the at least two oxylipins comprise Leukotriene B4, 19(20)-DiHDPA, 13(14)-DiHDPA, and DiHDPA. In still yet another embodiment of the method of treatment the at least two oxylipins comprise 9(S)-HODE, 16(17)-DiHDPA, 19(20)-EPDPA, 19(20)-DiHDPA, and 7(8)-DiHDPA.

In a certain embodiment of the method of treatment the at least two oxylipins comprise 13(S)-HODE, and 10(11)-EpDPA.

The method of treatment disclosed herein can comprise the testing of a subject biofluid sample wherein the biofluid sample is a blood sample, a serum sample, a plasma sample, a urine sample, a cerebrospinal fluid, and the like.

In certain embodiments the oxylipins can be detected by mass spectrometry (MS), nuclear magnetic resonance (NMR) spectroscopy, HPLC-UV, infrared spectroscopy, a biochemical assay, or an immunoassay in the biofluid sample.

The present disclosure also provides a method for predicting survival of a subject at high risk of CAD comprising detecting a threshold amount of a LA-derived oxylipin, an EPA-derived oxylipin, an ARA-derived oxylipin, or combinations thereof. In a particular embodiment the LA-derived oxylipin can comprises one or more of 13(S)-HODE, 10(11)-EpDPA, 9(S)-HODE, 5-HETE, 8-iso PGF3α, and thromboxane B2. In a more specific embodiment of the method the oxylipin can comprise a combination of 13(S)-HODE and 10(11)-EpDPA, or 9(S)-HODE and 10(11)-EpDPA.

In a certain embodiment of the method the subject does not require a coronary artery bypass graft (a CABG) and the oxylipin is 9(S)-HODE.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A and FIG. 1B depict biosynthetic pathways of plasma oxylipins from omega-6 and omega-3 polyunsaturated fatty acids (PUFA). In FIGS. 1A and 1B normal text designates PUFA and oxylipins. The italic text in oval boxes designates enzymes involved in metabolic transformation with their quantified oxylipins in boxes with the same cross-hatching and oxylipins below the limit of quantification (LOQ) in non-cross-hatched squares. See abbreviation index and appendix for full names description.

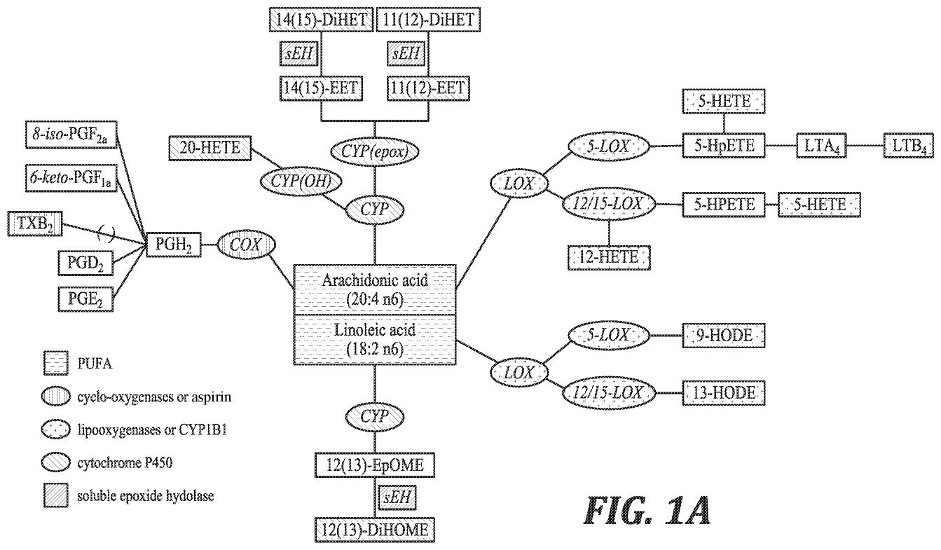

FIG. 2 depicts a study flow diagram of adults with obstructed coronary arteries (≥70% stenosis) from the greater Portland area, Oregon.

FIG. 3A through FIG. 3C depict diagnosis of the number of obstructed coronary arteries in adults with obstructed coronary arteries (≥70% stenosis; n=74), as shown by receiver operating characteristic (ROC) curves. FIG. 3A depicts a diagnosis of the number of obstructed coronary arteries based on the best single oxylipin model.

FIG. 3B depicts a diagnosis of the number of obstructed coronary arteries based the best single oxylipin group model. FIG. 3C depicts a diagnosis of the number of obstructed coronary arteries based on the smallest oxylipin panel model achieving AUC≥0.90.

FIG. 4A through FIG. 4C provide a prediction of survival during a 5-year follow up in adults with obstructed coronary arteries (≥70% stenosis; n=64); as shown by receiver operating characteristic (ROC) curves. FIG. 4A depicts a prediction of survival based on the best single oxylipin model. FIG. 4B depicts a prediction of survival based on the best single oxylipin group model. FIG. 4C depicts a prediction of survival based on the smallest oxylipin panel model achieving AUC≥0.90.

FIG. 5A through FIG. 5C depicts a prediction of survival without coronary artery bypass graft (CABG) surgery during a 5-year follow up in adults with obstructed coronary arteries (≥70% stenosis; n=64) as shown by ROC curves. FIG. 5A depicts a prediction of survival based on the best single oxylipin model. FIG. 5B depicts a prediction of survival based on the best single oxylipin group model. FIG. 5C depicts a prediction of survival based on the smallest oxylipin panel model achieving AUC≥0.85.

FIG. 6 depicts the link between oxylipins and coronary artery disease. Adults with more obstructed coronary arteries (≥70% stenosis) had lower plasma concentrations of hydroxylated omega-3 fatty acids derived epoxygenated oxylipins, which was linked to decreased soluble epoxide hydrolase (sEH) activity. Non-surviving adults with obstructed coronary arteries had higher plasma concentration of oxygenated omega-6 fatty acids, which was linked to increased lipoxygenase or CYP1B1 activity.

### DETAILED DESCRIPTION

This disclosure provides biomarkers useful for detecting and/or diagnosing the presence, extent and/or severity of coronary artery disease (CAD). The use of these biomarkers provide a vastly improved method for detecting and/or diagnosing the presence, extent and/or severity of coronary artery disease in at risk subject and can provide a method for determining the survivability of the condition without an invasive surgical procedure.

It has been found that concentrations of certain quantified oxylipins decreased with the number of obstructed arteries; a panel of five (5) oxylipins diagnosed three (3) obstructed arteries with 100% sensitivity and 70% specificity. Concentrations of five (5) oxylipins were lower and one (1) oxylipin was higher with survival; a panel of 2 oxylipins predicted survival during follow up with 86% sensitivity and 91% specificity. Plasma oxylipins can therefore assist in CAD diagnosis and prognosis alone or when used in combination with standard risk assessment tools.

### ABBREVIATIONS USED HEREIN

ACN: acetonitrile
ARA: arachidonic acid
CAD: coronary artery disease
COX: cyclooxygenases
CUDA: 1-cyclohexyl ureido, 3-dodecanoic acid
CVD: cardiovascular diseases
CYP450: cytochrome P450
CYPEPDX: CYP epoxide
CYPESE: CYP soluble epoxide
DHA: docosahexaenoic acid
EPA: eicosapentaenoic acid
FA: fatty acid
4-HNE: 4-hydroxynonenal
HPLC-MS/MS: high-performance liquid chromatography tandem mass spectrometry
IMS: ionization mass spectrometry
IPA: isopropanol
LA: linoleic acid
LDL: low-density lipoproteins
LNA: linolenic acid
LOD: limit of detection
LOQ: limit of quantification
LOX: lipoxygenases
MDA: malondialdehyde
MRM: multi-reaction monitoring
PUFA: polyunsaturated fatty acids
RT: retention time

### APPENDIX OF COMPOUNDS

8-iso PGF3a: 9S,11R,15S-trihydroxy-5Z,13E,17Z-prostatrienoate
Thromboxane B2: 9S,11,15S-trihydroxy-thromboxa-5Z
17(18)-DiHETE: 17(18)-dihydroxy-5Z,8Z,11Z,14Z-eicosatetraenoic acid
Leukotriene B4: (5S,6Z,8E,10E,12R,14Z)-5,12-dihydroxyicosa-6,8,10,14-tetraenoic acid
11(12)-DiHETE: 11(12)-dihydroxy-5Z,8Z,14Z,17Z-eicosatetraenoic acid
12(13)-DiHOME: 12(13)-di hydroxy-9Z-octadecenoic acid
5(6)-DiHETE: 5(6)-dihydroxy-8Z,11Z,14Z,17Z-eicosatetraenoic acid
19(20)-DiHDPA: 19(20)-dihydroxy-4Z,7Z,10Z,13Z,16Z-docosapentaenoic acid
14(15)-DiHET or 14(15)-DiHETrE: 14(15)-dihydroxy-5Z,8Z,11Z-eicosatrienoic acid
16(17)-DiHDPA: 16(17)-dihydroxy-4Z,7Z,10Z,13Z,19Z-docosapentaenoic acid
13(14)-DiHDPA: 13(14)-dihydroxy-4Z,7Z,10Z,16Z,19Z-docosapentaenoic acid
10(11)-DiHDPA: 10(11)-dihydroxy-4Z,7Z,13Z,16Z,19Z-docosapentaenoic acid
7(8)-DiHDPA: 7(8)-dihydroxydocosa-4Z,10Z,13Z,16Z,19Z-pentaenoic acid
20-HETE: 20-hydroxy-5Z,8Z,11Z,14Z-eicosatetraenoic acid
13(S)-HODE: 13S-hydroxy-9Z,11E-octadecadienoic acid
9(S)-HODE: 9S-hydroxy-10E,12Z-octadecadienoic acid
15-HETE: 15-hydroxy-5Z,8Z,11Z,13E-eicosatetraenoic acid
17(18)-EpETE: 17(18)-epoxy-5Z,8Z,11Z,14Z-eicosatetraenoic acid
12-HETE: 12-hydroxy-5Z,8Z,10E,14Z-eicosatetraenoic acid
5-HETE: 5-hydroxy-6E,8Z,11Z,14Z-eicosatetraenoic acid
19(20)-EpDPA or 19(20)-EpDPE: 19(20)-epoxy-4Z,7Z,10Z,13Z,16Z-docosapentaenoic acid
14(15)-EET or 14(15)-EpETrE: 14(15)-epoxy-5Z,8Z,11Z-eicosatrienoic acid
10(11)-EpDPA or 10(11)-EpDPE: (4Z,7Z)-9-[3-(2Z,5Z,8Z)-2,5,8-undecatrien-1-yl-2-oxiranyl]-4,7-nonadienoic acid
11(12)-EET or 11(12)-EpETrE: 11,(12)-epoxy-5Z,8Z,14Z-eicosatrienoic acid

Biomarkers of the present disclosure can be obtained from a subject in a variety of ways that are standard in the art. Although suitable subjects can include any person, it is preferred that the subject is a person with suspected coronary disease. By way of example, a subject can be suspected of having CAD if the subject suffers from symptoms associated with CAD, such as angina or chest pain, shortness, aching, burning, fullness, heaviness, numbness, pressure, squeezing, weakness, dizziness, faster heartbeat, nausea, palpitations, sweating, or fatigue. The symptoms are commonly felt in the chest, arm, back, jaw, neck or shoulder. Men and women may experience different symptoms. If the subject has greater than one risk factor for CAD, such as family history of premature coronary disease, smoking, high blood pressure, high cholesterol, diabetes, chronic kidney disease. Suitable subjects can also be determined based on age or other acceptable screening criteria. An additional example for determining whether a subject is in need of testing, having CAD, or at risk for CAD includes test results from, for example, a cardiac angiogram, echocardiogram, electrocardiogram, CT scan, or exercise stress test. A suitable subject for testing may be a subject age fifty or older.

Described herein is an evaluation of whether plasma oxylipins, alone or in panels, can diagnose the number of obstructed coronary arteries and evaluated whether they can also predict median 5-year outcomes in high CAD risk subjects with chest pain and ≥70% stenosis, which has apparently not been previously presented. The resultant evaluation of plasma oxylipins and panels of plasma oxylipins as presented herein provides a simple, inexpensive, point-of-care test that can assist in the decision process regarding subject management.

In the current disclosure, evidence is provided that in adults with obstructed coronary arteries with ≥70% stenosis, plasma oxylipin panels can diagnose the number of obstructed coronary arteries and predict median 5-year outcomes.

Methods of the disclosure comprise obtaining a sample from a human test subject. The present disclosure allows the concentration of the at least one oxylipin to be determined with only minimal processing of the biofluid sample obtained from the subject. Advantageously, the present methods allow for a simple and/or non-invasive (e.g., not requiring surgery or biopsy) and/or a quick and/or inexpensive processing a the biofluid sample.

Minimal processing of the biosample helps prevent the introduction of false positives and/or the provision of false negatives, especially when compared with other methods that may require concentration of the oxylipins.

The identification, or detecting of the level of concentration of the at least one oxylipin can be determined by any suitable method known to one of skill in the art. The identification or detecting of the concentration of the at least one oxylipin can be determined by one of more methods including mass spectrometry, including MALDI-TOF/MS, ESI-MS/MS, and GC-EI/MS, nuclear magnetic resonance (NMR) spectroscopy, HPLC-UV, thin layer chromatography, chiral chromatography, capillary electrophoresis, a biochemical assay, or an immunological assay. The immunological assay can include an ELISA assay or radioimmunoassay (RIA) wherein antibodies are used that are specific for at least each class or for each of the at least one oxylipins described herein. ELISA and RIA assays are well known in the art.

The sample obtained from the test subject or the at least one standard obtained from a subject not having or at risk for CAD can be a biological fluid (biofluid) sample, or a fraction thereof. The biofluid sample can be obtained by using any suitable technique known in the art including, for example, venipuncture, catheter extraction, lumbar puncture, urination, and the like. The biofluid sample can be a blood sample, a serum sample, a plasma sample, a cerebrospinal fluid sample, a lymph sample, a urine sample, a combination thereof or a fraction thereof. A preferred sample is a blood plasma sample.

The term “fraction thereof” as used herein in the context of a biofluid fraction refers to a portion of a biofluid sample obtainable or obtained following processing of the biofluid. A suitable biofluid fraction refers to one or more constituent(s) of the biofluid that has been separated from one of more additional biofluid constituents. For example, a fraction of a blood sample can be a blood plasma or blood serum fraction.

The term “reference standard” or “standard” refers to data on the level of concentration of the at least one oxylipins obtained from a subject, preferably a human subject, of a known diagnostic status. For example, the standard is obtained from a subject known to not be at risk and/or have CAD. Suitably, the data on oxylipin concentrations of levels obtained from the reference or standard subject are obtained using similar and preferably identical techniques to those used to determine the level and/or concentration of the at least one oxylipin for the test subject. The “reference standard” sample is obtained from the same type of biofluid sample as the sample obtained from the test subject.

Current analytical methods for extraction, detection, and data processing allow for the separation of a large number of diverse oxylipins in a short time period (Neyeem, 2018; Tourdot et al., 2014; Pedersen and Newman, 2018; La Frano et al., 2017). Disclosed herein, 39 oxylipins of diverse origin and biosynthetic pathways were detected and verified with standards in a 22-min run. Similar to inflammatory cytokines, low abundance, limited dynamic range, limited tissue specificity, very short half-life, significant daily fluctuation, and high inter- and intra-assay variation, have limited the use of oxylipins as diagnostic biomarkers (Strassburg et al., 2012). For diagnostic and prognostic research, a good biomarker must have a large dynamic range within the population. As determined herein, 22 oxylipins had concentrations in the linear quantification range in at least 98% of sampled adults, which allowed for the evaluation of the most abundant enzymatic oxylipin pathways; however, excluded pathways generated by COX or aspirin and ROS.

### Diagnostic and Prognostic Efficacy of Oxylipins in CAD

Currently used risk assessment scores of CAD, such as the 10-year Framingham general cardiovascular disease (CVD) risk score, have been developed for the general population, and have shown limited efficacy in high risk CAD adult management (Hajar, 2017). In adults with obstructed coronary arteries, a five-oxylipin panel determined and/or diagnosed three (3) obstructed arteries with 100% sensitivity and 70% specificity. During median 5-year survival, a panel of two (2) oxylipins identified herein predicted survival during follow-up with 86% sensitivity and 91% specificity. The oxylipin panels determined herein improved three (3) obstructed artery (CADS) diagnosis and survival prognosis compared to the 10-year Framingham general CVD risk score.

In the present disclosure, a combination of HPLC and quantitative tandem mass spectrometry was used to quantify oxylipins. To serve as point-of-care biomarker, oxylipin analysis an alternative diagnostic method adapted to another method, such as an ELISA or an RIA is provided. In addition, further validation in a larger unselected population easily accomplished using the methods provided herein.

### Clinical Relevance of Oxylipins in CAD

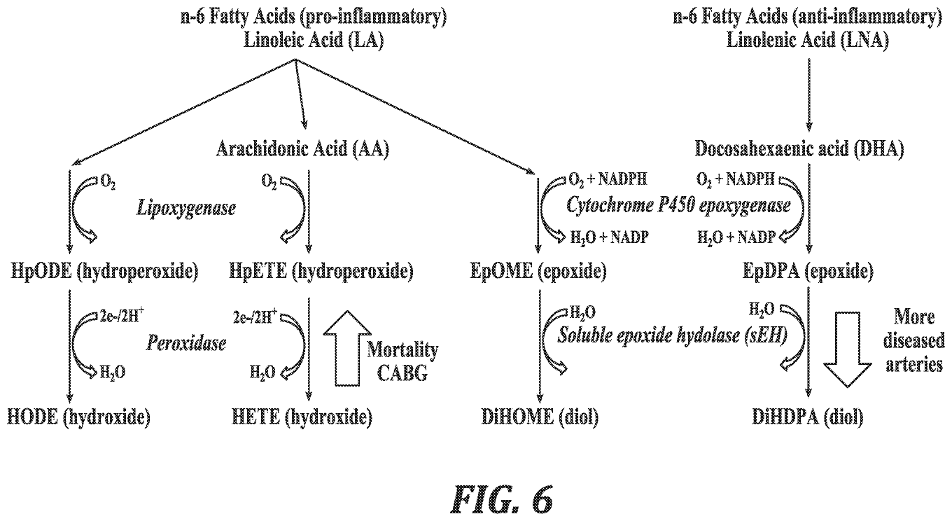

Coronary artery disease (CAD) limits nutrient and oxygen supply to generate sufficient energy in cardiomyocytes, which becomes an even bigger challenge as the number of occluded coronary arteries increases or plaques and thrombosis rupture or get dislodged (Ambrose and Singh, 2015). Disclosed herein, adults with more obstructed coronary arteries (≥70% stenosis) had lower plasma concentrations of hydroxylated omega-3 PUFA derived DHA-derived epoxides, specifically inhibition of hydroxylation of 19(20)-EpDPA to 19(20)-DiHDPA (FIG. 6). This link between oxylipin concentrations and the number of obstructed CAD has not been previously reported. The enzyme responsible for hydroxylation of epoxides is sEH, which is induced by vascular hypoxia and has been proposed as potential pharmacological target for CAD (Morisseau and Hammock, 2012; Imig, 2018; Wagner et al., 2017). The possibility cannot be excluded that participants with CAD were already on medication that inhibited soluble CYP450 epoxide hydrolase. However, the gradual decrease in concentrations of LA-derived 12(13)-DiHOME and DHA-derived DiHDPA with increasing obstructed artery number support the hypothesis that the lower concentrations are a response to the hypoxia caused by the arterial occlusions (Fleming, 2014).

Five-year survival and no open-heart CABG surgery was linked to concentrations of oxygenated LA and ARA, specifically lower concentrations of LA-derived mid-chain HODE and ARA-derived mid-chain HETE, which are either generated by oxygenation of lipoxygenases or hydroxylation of CYP1B1 (FIG. 6). In support, elevated 15-HETE concentrations and LOX-15 enzymatic activity have been reported in ischemic heart disease and hypoxic human cardiomyocytes and cardiac endothelial cells (Lundqvist et al., 2016), supporting the hypothesis that the elevated mid-chain HETE and HODE concentrations are a response to the hypoxia caused by the arterial occlusions. High concentrations of HETE, including 5-HETE, 12-HETE, and 15-HETE, were reported in atherosclerotic plaques, especially in those that were more likely to rupture (Mallat et al., 1999). Elevated circulating concentrations of 5-HETE and 12-HETE were observed in individuals after cardiac surgery (Strassburg et al., 2012). Elevated concentrations of 5-HETE, 12-HETE, and 15-HETE were reported in individuals with acute cardiac syndrome (Zu et al., 2016). Elevated circulating concentrations of 5-HETE, 12-HETE, and 15-HETE were reported in individuals with CAD by Xu et al. (2013), whereas only numerical increases were reported by Shishebor et al. (2006) and Auguet et al. (2018); the latter did not quantify 5-HETE. The role of elevated mid-chain HETE in cardiovascular dysfunction has been well documented, whereas less is known of the role of mid-chain HODE (Nayeem, 2018; Dobrian et al., 2011; Maayah and El-Kadi, 2016). Inhibition of the oxygenation step of the LOX pathway has been proposed as treatment option for CAD management (Dobrian et al., 2011), suggesting clinical relevance of the identified oxylipins as an indicator of chronic vascular hypoxia.

In one embodiment of the present method a panel of 6 oxylipins is provided. The six oxylipin panel can be used to detect or determine whether a subject is in a CAD state, a maximum threshold for the oxylipins wherein concentrations above the threshold indicate a CAD state. The results from the panel can be used in choosing a type of invasive surgery, whether the subject is likely to survive without CABG or with CABG. The five oxylipin panel comprises 9(S)-HODE, 16(17)-DiHDPA, 19(20)-EPDPA, 19(20)-DiHDPA, and 7(8)-DiHDPA. The threshold amounts of each of the five oxylipins above which a CAD state is indicated are <12.7 nM 9(S)-HODE, <0.39 nM 16(17)-DiHDPA, <0.38 nM 19(20)-EPDPA, 2.3 nM 19(20)-DiHDPA, or 0.151 nM. 7(8)-DiHDPA.

In another embodiment comprising a 4 oxylipin panel is provided, the panel is useful to determine a CAD state when the concentration of the oxylipin is below a minimum threshold, and when the concentration of the oxylipin is above a maximum threshold. The panel in particular can be used to determine whether surgery is an option for the subject and/or for choosing a type of invasive surgery to treat the subject suffering from CAD, and to determine whether a subject has 3 diseased arteries or one or two diseased arteries. The oxylipins that make up the panel comprise leukotriene B4, 19(20)-DiHDPA, 12(14)-DiHDPA, and 10(11)-DiHDPA. The maximum threshold values for the oxylipins of the panel of 4 oxylipins comprise <0.211 nM leukotriene B4, <2.18 nM 19(20)-DiHDPA, >0.09 nM 13(14)-DiHDPA, or between 0.041 to 0.08 nM 10(11)-DiHDPA.

In another embodiment a panel that comprises 2 oxylipins is provided. Detection and quantification of the 2 oxylipins is useful to determine a CAD state when the concentrations of each oxylipin is lower than found in a subject without CAD, and wherein the concentration of the 2 oxylipins also provides a maximum threshold above which a CAD state is indicated. In a particular embodiment the panel can be used to predict the outcome of an intervention. The ultimate outcome can include death, wherein the intervention would be considered ineffective. In addition, the panel can be used to predict survival versus death in a subject with CAD and whether an intervention is required to potentially prevent death. The oxylipins that make up the panel comprise 13(S)-HODE and 10(11)-EpEPA. The threshold amounts of the oxylipins above which a CAD state is indicated comprise >42.5 nM 13(S)-HODE and <0.20 nM 10(11)-EpDPA.

In summary, in spite of the certain limitations to the studies described herein a link between plasma oxylipin concentrations and CAD severity has definitively been determined. In certain embodiments the concentrations of six (6) oxylipins decreased with number of obstructed arteries; a panel of five (5) oxylipins was capable of diagnosing subjects with three (3) obstructed arteries with 100% sensitivity and 70% specificity. In certain other embodiments it was found that the concentrations of five (5) oxylipins were lower and one (1) oxylipin was higher with increased survival of the subjects. In still another embodiment it was found that a panel of two (2) oxylipins predicted survival during follow-up with 86% sensitivity and 91% specificity. Therefore, plasma oxylipins can be used in diagnosis and prognosis of CAD in high-risk adults and can assist is such diagnosis and prognosis of CAD in high-risk adults when used alone or in combination with standard risk assessment tools. The methods disclosed provide greatly improved methods for the diagnosis and prognosis of CAD. The improvements comprise a method which is simpler, less expensive, and more reliable than methods currently available in the prior art.

### EXAMPLES

### Oxylipins Linked to CAD

This example demonstrates that oxylipin levels can discriminate between individuals with and without CAD and provide an indication of the extent of CAD. Hence, oxylipin levels are important prognosticators for adverse cardiovascular outcomes. Using oxylipin levels as biomarkers for CAD presence/absence or extent may fundamentally change the way in which we screen for CAD.

### Materials and Methods

### Participants and Study Design

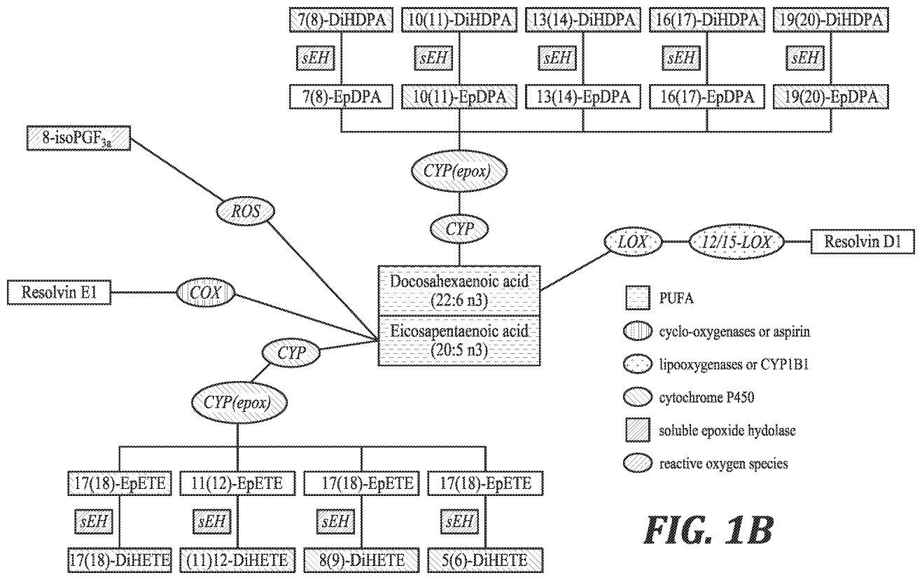

For the CAD groups, 74 individuals from the greater Portland metropolitan area were prospectively enrolled from October 2012 and January 2017 who were referred to OHSU, Portland (Oreg.), for a CT coronary angiogram because of suspicious chest pain or angina (median age: 66 years; range 38 to 87 years). Inclusion criteria were a inducible myocardial ischemia during stress (either on echocardiography or single-photon computed tomography) and ≥70% coronary luminal narrowing of one or more major coronary artery or its major branches on subsequent coronary angioplasty. Exclusion criteria were <70% coronary stenosis on angiography, prior myocardial infarction, hemodynamically significant valvular heart disease, prior re-vascularization or congestive heart failure. The CAD patients were classified as having 1-vessel (n=31), 2-vessel (n=23); or 3-vessel (n=20) CAD and were followed until November 2019 (FIG. 2) for a median of 60 months (range 25 to 84 months) for adverse events (i.e., coronary stent placement; coronary artery bypass graft (CABG) surgery; death). Ten CAD patients were lost to follow up (unable to contact: n=8; declined to follow up: n=2).

To establish ranges of plasma oxylipin concentration in low CAD risk populations, an Astoria cohort was enlisted and 220 individuals were prospectively enrolled from July 2016 to February 2017. For the study disclosed herein, individuals of the same age range (range: 38-71 years) that had the lowest CAD risk scores (n=23). Exclusion criteria were self-reported history of hypertension, hyperlipidemia, diabetes, myocardial infarction, ischemia, coronary angiography, active tobacco use, and a family history of CAD. The Institutional Review Board of Oregon Health and Science University approved the study.

### Sample Collection and Preparation for Oxylipin Analysis

All participants fasted for at least 6 hr before 4.5 mL blood was collected in tubes containing 0.01 M buffered sodium citrate and immediately placed on ice. Blood samples were collected 1 to 4 hr prior to coronary angiography of participants with CAD. Whole blood samples were then centrifuged at 3,000 rpm for 15 min in a refrigerated centrifuge at 4° C., after which the plasma was aliquoted into 1 mL Eppendorf tubes and immediately stored at −80° C. until analysis.

Oxylipins from plasma were extracted as described in Perdersen et al. (2018) with minor modifications (Garcia-Jaramillo et al., 2019a,b). In brief, plasma samples (200 μL) were placed into 1.5 mL polypropylene tubes containing 7.5 μL anti-oxidant solution (0.2 mg mL⁻¹ solution butylated hydroxytoluene (BHT) in ethanol), and 3 μL of a deuterated internal standard solution (prepared with a combination of 20 deuterated oxylipins in ethanol at a concentration of 5 ngμL⁻¹) each one, was added. Solutions were transferred into a 96-well Ostro Pass Through Sample Preparation Plate (Waters Corp, Milford, Mass., USA). 450 μL of an acetonitrile solution containing 1% formic acid was vigorously added to each well and the mixture aspirated 3 times. The solution was eluted into glass inserts containing 10 μL methanol (containing 10% glycerol) by applying vacuum at 15 Hg for 10 min. Eluents were dried by vacuum centrifugation in a centrifugal vacuum concentrator (Labconco Centrivap®; Kansas City, Mo.) for 1 hr at 30° C. Once dry, samples were re-constituted with 100 μL of methanol:acetonitrile (50:50), containing the internal standard CUDA (12-[[(cyclohexylamino)carbonyl]amino]-dodecanoic acid) (Cayman Chemical, Ann Arbor, Mich.) at 50 ng mL⁻¹. Samples were mixed vortex for 1 min, transferred to a spin filter (0.22 μm PVDF membrane, Millipore-Sigma, Burlington, Mass., USA), and centrifuged for 3 min at 6° C. at 9,000 rpm, before to be transferred to 2 mL LC-MS amber vials. Extracts were stored at −20° C. until analysis (less than 48 h) by high-performance liquid chromatography tandem mass spectrometry (HPLC-MS/MS). The internal oxylipin standards (Table 1) used during the extraction were used to correct the recovery of the quantified oxylipins (La Frano et al., 2017).

TABLE 1
Detailed list of multi-reaction monitoring (MRM) transitions for the deuterated-oxylipins
(surrogates) and CUDA (12-[[(cyclohexylamino) carbonyl] amino]-dodecanoic acid)
used as internal standards for our analysis. Compounds are ordered based on retention time (RT).

| | Precursor | Product | | | | | LOD | LOQ |
|---|---|---|---|---|---|---|---|---|
| Surrogotes | Ion | Ion | RT | DP | CE | S/N | (ng/μL) | (ng/μL) |
| 6 keto PGF1α - d4 | 373 | 167 | 6.0 | −70 | −40 | 5.7 | 0.084 | 0.281 |
| Resolvin E1 - d4 | 353 | 197 | 6.0 | −40 | −20 | 11.4 | 0.42 | 0.140 |
| Thromboxane B2 - d4 | 373 | 173 | 7.1 | −50 | −21 | 24.6 | 0.020 | 0.065 |
| PGF2a - d4 | 357 | 197 | 7.6 | −70 | −33 | 2.8 | 0.171 | 0.571 |
| PGE2 - d4 | 355 | 275 | 7.9 | −35 | −25 | 43.6 | 0.011 | 0.037 |
| PGD2 - d4 | 355 | 275 | 8.2 | −35 | −25 | 65.3 | 0.007 | 0.025 |
| Resolvin D1 - d5 | 380 | 141 | 8.7 | −40 | −20 | 1.5 | 0.320 | 1.067 |
| Leukotriene B4 - d4 | 339 | 197 | 11.6 | −70 | −30 | 60.8 | 0.008 | 0.026 |
| CUDA | 339 | 214 | 12.1 | −65 | −35 | 1310.3 | 0.000 | 0.001 |
| 12(13)-DiHOME - d4 | 317 | 185 | 12.3 | −70 | −30 | 52.3 | 0.009 | 0.031 |
| 20-HETE - d6 | 325 | 281 | 14.5 | −65 | −24 | 3.1 | 0.155 | 0.516 |
| 13(S)-HODE - d4 | 299 | 198 | 15.3 | −65 | −25 | 30.5 | 0.016 | 0.053 |
| 9(S)-HODE - d4 | 299 | 172 | 15.4 | −70 | −16 | 5.3 | 0.091 | 0.302 |
| 15(S)-HETE - d8 | 327 | 226 | 15.5 | −70 | −16 | 63602.5 | 0.000 | 0.000 |
| 12(S)-HETE - d8 | 327 | 184 | 16.0 | −60 | −21 | 15 | 0.032 | 0.107 |
| 5(S)-HETE - d8 | 327 | 116 | 26.4 | −50 | −20 | 35.6 | 0.014 | 0.045 |
| 14(15)-EET_(EpETrE) - d11 | 330 | 175 | 16.8 | −70 | −16 | 48.3 | 0.010 | 0.033 |
| 11(12)-EET_(EpETrE) - d11 | 330 | 167 | 17.1 | −55 | −15 | 8.8 | 0.055 | 0.182 |
| EPA - d5 | 306 | 262 | 28.4 | −55 | −20 | 5.4 | 0.089 | 0.296 |
| DHA - d5 | 332 | 234 | 18.4 | −55 | −20 | 10.5 | 0.046 | 0.152 |
| ARA - d8 | 311 | 267 | 18.6 | −60 | −18 | 15.2 | 0.032 | 0.105 |

RT: retention time Imin);
DP: declustering potential (V);
CE: collision energy (V);
S/N: signal to noise ratio;
LOD: limit of detection;
LOQ: limit of quantification.

### Chromatographic and Mass Spectrometric Analysis of Oxylipins

High Performance Liquid Chromatography (HPLC) was performed using a Shimadzu system (Shimadzu, Columbia, Md.) coupled to a 4000 QTRAP® LC-MS/MS system (AB SCIEX, Framingham, Mass.) as previously described in Garcia-Jaramillo et al. (2019a,b). Employing dynamic multi-reaction monitoring (dMRM) 60 oxylipins were evaluated in a targeted approach. For each compound, optimal transitions were determined by flow injection of pure standards using the optimizer application, and transitions were compared to literature when available for the certain compounds. The detailed list of MRM transitions is found in Table 2.

TABLE 2
Detailed list of multi-reaction monitoring (MRM) transitions for the oxylipins contained
in an in-house library. Compounds are ordered based on retention time (RT).

| | | | Precursor | Product | | | | | LOD | LOQ |
|---|---|---|---|---|---|---|---|---|---|---|
| Precursor | Pathway | Compound | Ion | Ion | RT | DP | CE | S/N | ng/pL | ng/pL |
| C20:4 | COX | 6-keto PGF1α | 369 | 163 | 6.0 | −70 | −40 | 21.3 | 0.023 | 0.075 |
| C20:5 | COX | Resolving E1 | 349 | 195 | 6.1 | −40 | −20 | 6.7 | 0.072 | 0.239 |
| C20:5 | ROS | 8-iso PGF3α | 351 | 307 | 6.3 | −80 | −26 | 11.4 | 0.042 | 0.140 |
| C20:4 | COX | 8-iso PGF2α | 353 | 193 | 7.1 | −70 | −33 | 5.9 | 0.081 | 0.271 |
| C20:4 | COX | Thromboxane B2 | 369 | 169 | 7.2 | −50 | −21 | 309.7 | 0.002 | 0.005 |
| C20:4 | COX | PGE2 | 351 | 271 | 7.9 | −35 | −25 | 651.8 | 0.001 | 0.003 |
| C20:4 | COX | PGD2 | 351 | 271 | 8.3 | −35 | −25 | 505.8 | 0.001 | 0.003 |
| C20:6 | LOX | Resolvin D1 | 375 | 121 | 8.8 | −40 | −20 | 10 | 0.048 | 0.160 |
| C22:6 | LOX | PDX | 359 | 153 | 11.3 | −20 | −20 | 6186.1 | 0.000 | 0.000 |
| C20:5 | CYPEPOX/sEH | 17(18)-DiHETE | 335 | 203 | 11.4 | −60 | −22 | 3689.7 | 0.000 | 0.000 |
| C20:4 | LOX5 | Leukotriene B4 | 335 | 195 | 11.6 | −70 | −21 | 2163.1 | 0.000 | 0.001 |
| C20:5 | CYPEPOX/sEH | 14(15)-DiHETE | 335 | 111 | 11.8 | −55 | −22 | 810.4 | 0.001 | 0.002 |
| C20:5 | CYPEPOX/sEH | 11(12)-DiHETE | 335 | 167 | 12.0 | −55 | −22 | 1380.4 | 0.000 | 0.001 |
| C20:5 | CYPEPOX/she | 8(9)-DiHETE | 335 | 185 | 12.3 | −55 | −22 | 7 | 0.069 | 0.229 |
| C18:2 | CYPEPOX/sEH | 12(13)-DiHOME | 313 | 183 | 12.3 | −70 | −30 | 1262.4 | 0.000 | 0.001 |
| C20:5 | CYPEPOX/she | 5(6)-DiHETE | 335 | 145 | 12.9 | −55 | −22 | 17.7 | 0.027 | 0.090 |
| C22:6 | CYPEPOX/she | 19(20)-DiHDPA | 361 | 229 | 12.9 | −74 | −24 | 1.3 | 0.369 | 1.231 |
| C20:4 | CYPEPOX/she | 14(15)-DiHET | 337 | 207 | 13.0 | −65 | −25 | 38.8 | 0.012 | 0.041 |
| C22:6 | CYPEPOX/she | 16(17)-DiHDPA | 361 | 233 | 13.3 | −80 | −24 | 8.6 | 0.056 | 0.186 |
| C22:6 | CYPEPOX/she | 13(14)-DiHDPA | 361 | 193 | 13.6 | −80 | −24 | 39.9 | 0.012 | 0.040 |
| C22:6 | CYPEPOX/sEH | 10(11)-DiHDPA | 361 | 153 | 13.8 | −80 | −24 | 23.4 | 0.021 | 0.068 |
| C22:6 | CYPEPOX/she | 7(8)-DiHDPA | 361 | 127 | 14.4 | −80 | −24 | 25.8 | 0.019 | 0.062 |
| C20:4 | CYPOH | 20-HETE | 319 | 245 | 14.6 | −65 | −24 | 42.7 | 0.011 | 0.038 |
| C18:2 | LOX12/15 | 13(S)-HODE | 295 | 195 | 15.4 | −65 | −25 | 2871 | 0.000 | 0.001 |
| C18:2 | LOX5 | 9(S)-HODE | 295 | 171 | 15.5 | −60 | −25 | 2743.5 | 0.000 | 0.001 |
| C20:4 | LOX12/15 | 15-HETE | 319 | 175 | 15.7 | −70 | −16 | 8.7 | 0.055 | 0.184 |
| C20:5 | CYPEPOX | 17(18)-EpETE | 317 | 215 | 15.7 | −55 | −15 | 34.8 | 0.014 | 0.046 |
| C20:5 | CYPEPOX | 14(15)-EpETE | 317 | 248 | 16.0 | −45 | −15 | 7754.4 | 0.000 | 0.000 |
| C20:5 | CYPEPOX | 11(12)-HpETE | 317 | 195 | 16.1 | −70 | −16 | 17.3 | 0.028 | 0.093 |
| C20:4 | LOX12/15 | 12-HETE | 319 | 135 | 16.1 | −60 | −21 | 50.3 | 0.010 | 0.032 |
| C20:5 | CYPEPOX | 8(9)-EpETE | 317 | 155 | 16.3 | −75 | −16 | 3721.6 | 0.000 | 0.000 |
| C20:4 | LOX5 | 5-HETE | 319 | 115 | 16.5 | −50 | −20 | 80.6 | 0.006 | 0.020 |
| C22:6 | CYPEPOX | 19(20)-EpDPA | 343 | 241 | 16.7 | −45 | −20 | 11.4 | 0.042 | 0.140 |
| C22:4 | CYPEPOX | 14(15)-EET | 319 | 175 | 16.9 | −70 | −16 | 1924.7 | 0.000 | 0.001 |
| C20:4 | CYPEPOX | 16(17)-EpDTA | 343 | 274 | 16.9 | −55 | −15 | 55832.9 | 0.000 | 0.0000 |
| C22:6 | CYPEPOX | 10(11)-EpDPA | 343 | 153 | 17.0 | −55 | −15 | 152.8 | 0.003 | 0.011 |
| C22:6 | CYPEPOX | 13(14-EpDPA | 343 | 161 | 17.2 | −55 | −15 | 2.5 | 0.192 | 0.640 |
| C20:4 | CYPEPOX | 11(12)-EET | 319 | 167 | 17.2 | −55 | −15 | 985.6 | 0.001 | 0.002 |
| C22:6 | CYPEPOX | 7(8)-EpDPA | 343 | 113 | 17.2 | −55 | −15 | 11.5 | 0.042 | 0.139 |

RT: retention time (min);
DP: declustering potential;
CE: collision energy (V);
S/N: signal to Noise ratio;
LOD: limit of detection;
LOQ: limit of quantification.

Compounds were separated using a Waters™ Acquity UPLC CSH C18 column (100 mm length×2.1 mm id; 1.7 μm particle size) with an additional Waters Acquity VanGuard CSH C18 pre-column (5 mm×2.1 mm id; 1.7 μm particle size) held constant at 60° C. The mobile phase consisted of (A) water (0.1% acetic acid) and (B) acetonitrile/isopropanol (ACN/IPA) (90/10, v/v) (0.1% acetic acid). Gradient elution (Pedersen et al., 2018) was carried out for 22 min at a flow rate of 0.15 mL min⁻¹. Gradient conditions were as follows: 0 to 1.0 min, 0.1 to 25% B; 1.0 to 2.5 min, 25 to 40% B; 2.5 to 4.5 min, 40 to 42% B; 4.5 to 10.5 min, 42 to 50% B; 10.5 to 12.5 min, 50 to 65% B; 12.5 to 14 min, 65 to 75% B; 14 to 14.5 min, 75 to 85% B; 14.5 to 20 min, 85 to 95% B; 20 to 20.5 min, 95 to 95% B; 20.5 to 22 min, 95 to 25% B. A 5 μL aliquot of each sample was injected onto the column. Limits of detection (LOD) and quantification (LOQ) (Table 1) were calculated based on one concentration point (0.1 ng μL⁻¹) for each oxylipin and deuterated surrogate.

### Data Processing and Statistical Analysis

Raw data from targeted oxylipin analyses were imported into MultiQuant software (AB SCIEX) to perform the alignment and integration of the peaks (obtaining peak areas). This software allowed for the correction of metabolite intensity with the intensity of the internal standards. Data obtained with MultiQuant were imported into MarkerView software (AB SCIEX) for initial data visualization (Housley et al., 2018).

Data were analyzed using SAS version 9.2 (SAS Ins. Inc., Cary, N.C.). Demographic and clinical characteristics of groups were compared using Fisher's exact test for binary data and t-test for non-binary data. Oxylipin concentrations were compared using Wilcoxon rank sum test. To evaluate diagnostic and predictive efficacy of oxylipins, logistic regression analysis was used and calculated receiver operatory characteristic (ROC) values, including area under the curves (AUC). The goal was to identify oxylipin panels that could achieve an ROC of 0.90 or higher. To compare diagnostic and predictive efficacy of oxylipins with a current standard risk assessment tool, ROC values of the best oxylipin models were compared with those of the 10-year Framingham general CVD risk scores. The 10-year atherosclerotic CVD risk score of the American College of Cardiology (ACC) was not used because 41 of 74 CAD patient scores could not be calculated. All statistical tests were two-sided. Significance was declared at p≤0.05.

### Results

### Oxylipin Analysis

In order to achieve a representative coverage of LA-, ARA-, EPA- and DHA-derived oxylipins and the enzymatic and non-enzymatic pathways involved in their production, a library with standards of 39 oxylipins was analyzed (Table 1). All 39 oxylipins were detected in one 22-min run. Of the 39 oxylipins, 24 were consistently above the LOD, and 22 oxylipins were consistently (i.e., below the LOQ in <3 adults) above the LOQ. Oxylipin concentrations below the LOQ were set at 80% of the lowest quantifiable sample. The library included (i) 4 LA-derived oxylipins (2 each from CYP450 and LOX pathways), of which 3 (CYP450: 12(13)-DiHOME, LOX: 9(S) HODE, and 13(S) HODE) were above the LOQ; (ii) 14 ARA-derived oxylipins (5 from COX, 5 from CYP450, and 4 from LOX pathways), of which 9 (COX: thromboxane B2; CYP450: 11(12)-EET, 14(15)-EET, 20-HETE, and 14(15)-DiHET; LOX: 5-HETE, 12-HETE, 15-HETE, and leukotriene B4) were above the LOQ; (iii) 10 EPA-derived oxylipins (1 from COX, 8 from CYP450, and 1 from ROS pathways), of which 3 (CYP450: 11(12)-DiHETE, and 17(18)-EpETE; and ROS: 8-iso PGF3a) were above the LOQ; (iv) 11 DHA-derived oxylipins (10 from CYP450 and 1 from LOX pathways) of which 7 (CYP450: 10(11)-EpDPA, 19(20)-EpDPA, 7(8)-DiHDPA, 10(11)-DiHDPA, 13(14)-DiHDPA, 16(17)-DiHDPA, and 19(20)-DiHDPA) were above the LOQ.

### Diagnosis of Number of Obstructed Coronary Arteries in Adults

Selected demographic and clinical characteristics of adults with obstructed coronary arteries stratified by number of diseased arteries and adults of the same age range with a low CAD risk are listed in Table 3. Sixty-nine (69) of seventy-four (74) adults with CAD had multiple CAD risk factors (3 CAD1 patients and 1 CAD2 patient had one CAD risk factor and one CAD2 patient had no CAD risk factor). Almost all adults with CAD had hypertension and hypercholesterolemia. Most adults with CAD were on aspirin, were overweight or obese, or had a history of smoking. About half adults with CAD had diabetes or a family history of CVD. Demographic and clinical characteristics of adults with CAD had a limited efficacy to diagnose number of obstructed arteries. The 10-year Framingham general CVD risk score and the number of CAD risk factors increased with number of obstructed arteries; specifically, adults with multiple diseased arteries were more likely to be male, were overweight or obese, former smokers, or had lower plasma HDL-cholesterol concentrations.

TABLE 3
Demographic and clinical characteristics of adults with obstructed coronary arteries (≥70%
stenosis) and adults of the same age range with a low coronary artery disease (CAD) risk.

| | Low | | Number of obstructed arteries | | | | | | Contrast |
|---|---|---|---|---|---|---|---|---|---|
| | CAD Risk | | One | | Two | | Three | | CAD1/2 |
| | (n = 23) | | (n = 31) | | (n = 23) | | (n = 20) | | vs. CAD3 |
| Characteristics | Mean ± STD | | Mean ± STD | | Mean ± STD | | Mean ± STD | | P-value |
| Age (yr) | 49 ± 10^{b} | | 65 ± 9^{a} | | 67 ± 12^{a} | | 66 ± 1^{a} | | 0.89 |
| Male, n (%) | 4 | (17)^{c} | 17 | (55)^{b} | 19 | (83)^{a} | 17 | (85)^{a} | 0.15 |
| BMI (kg/m²) | 28.3 ± 6.7^{ab} | | 28.1 ± 4.9^{b} | | 31.0 ± 9.3^{ab} | | 31.7 ± 6.1^{a} | | 0.20 |
| Overweight | 3 | (13)^{ab} | 15 | (48)^{a} | 9 | (39)^{ab} | 8 | (40)^{ab} | 0.80 |
| Obese | 9 | (39) | 8 | (26) | 10 | (43) | 10 | (50) | 0.28 |

Blood Pressure (mm Hg)

| | | | | | |
|---|---|---|---|---|---|
| Systolic | 124 ± 9 | 131 ±20 | 133 ± 17 | 129 ± 17 | 0.58 |
| Diastolic | 79 ± 6^{a} | 71 ± 13^{b} | 69 ± 11^{b} | 70 ± 11^{b} | 0.89 |

Plasma:

| Triacylglycerol (mg/dL) | 88 ± 38^{b} | 136 ± 72^{a} | | 165 ± 106^{a} | | 218 ± 326^{ab} | | 0.40 |
|---|---|---|---|---|---|---|---|---|
| Total Cholesterol (mg/dL) | 195 ± 32 | 182 ± 49^{a} | | 156 ± 33^{b} | | 177 ± 60^{ab} | | 0.62 |
| HDL-Cholesterol (mg/dL) | 65 ± 13^{a} | 52 ± 16^{b} | | 46 ± 14^{b} | | 40 ± 12^{c} | | 0.03 |
| LDL-Cholesterol (mg/dL) | 126 ± 28^{a} | 101 ± 35^{b} | | 76 ± 25^{b} | | 104 ± 48^{ab} | | 0.28 |
| Hbalc (mmol/mol) | 5.3 ± 0.4^{b} | 6.1 ± 1.1^{a} | | 6.5 ± 1.3^{a} | | 6.3 ± 1.1^{a} | | 0.90 |
| Medication: | 0^{b} | 30 | (97)^{a} | 21 | (91)^{a} | 18 | (90)^{a} | 0.61 |
| Blood Pressure, Total, n(%) | 0^{b} | 24 | (77)^{b} | 21 | (91)^{ab} | 20 | (100)^{a} | 0.10 |
| ACE Inhibitor, n (%) | 0^{b} | 10 | (32)^{a} | 8 | (35)^{a} | 5 | (25)^{a} | 0.58 |
| Angiotension Receptor Blocker | 0^{b} | 5 | (16)^{ab} | 3 | (13)^{ab} | 5 | (25)^{a} | 0.32 |
| Beta Blocker, n (%) | 0^{b} | 18 | (58)^{a} | 19 | (83)^{a} | 13 | (65)^{a} | 0.79 |
| Calcium Channel Blocker, n (%) | 0^{b} | 7 | (23)^{a} | 4 | (17)^{ab} | 5 | (25)^{a} | 0.75 |
| Diabetes, Total, n (%) | 0^{b} | 6 | (19)^{a} | 6 | (26)^{a} | 6 | (30)^{a} | 0.55 |
| Oral Hyperglycemia, n (%) | 0^{b} | 3 | (10)^{ab} | 3 | (13)^{ab} | 4 | (20)^{a} | 0.44 |
| Insulin, n (%) | 0 | 3 | (10) | 3 | (13) | 2 | (10) | 1 |

Hyperlipidemia, Total

| | | | | | | | | |
|---|---|---|---|---|---|---|---|---|
| Statin, n (%) | 0^{b} | 24 | (77)^{a} | 16 | (70)^{a} | 13 | (65)^{a} | 0.56 |
| Aspirin, n (%) | 0^{b} | 23 | (74)^{a} | 19 | (83)^{a} | 15 | (75)^{a} | 0.77 |

CVD Risk Factors:
Tobacco Use, n (%)

| | | | | | | | | | |
|---|---|---|---|---|---|---|---|---|---|
| Former | 6 | (26) | 8 | (26) | 9 | (39) | 6 | (30) | 1 |

| | | | | | | | | |
|---|---|---|---|---|---|---|---|---|
| Active | 0^{b} | 4 | (13)^{ab} | 2 | (9)^{ab} | 5 | (25)^{a} | 0.15 |

History of, n (%):

| | | | | | | | | | |
|---|---|---|---|---|---|---|---|---|---|
| Hypertension | 2 | (9)^{b} | 25 | (81)^{a} | 22 | (96)^{a} | 20 | (100)^{a} | 0.18 |

| | | | | | | | | |
|---|---|---|---|---|---|---|---|---|
| Diabetes | 0^{b} | 8 | (26)^{a} | 8 | (49)^{a} | 9 | (45)^{a} | 0.27 |
| Hypercholesterolemia | 0^{b} | 30 | (97)^{a} | 21 | (91)^{a} | 19 | (95)^{a} | 1 |
| CVD in Family | 0^{b} | 14 | (45)^{a} | 12 | (52)^{a} | 11 | (55)^{a} | 0.79 |

| | | | | | |
|---|---|---|---|---|---|
| Total risk, (1-5) | 0^{c} | 2.6 ± 0.9^{b} | 2.8 ± 0.9^{ab} | 3.2 ± 0.8^{a} | 0.04 |
| Framingham 10-yr CVD Risk (%) | 4.0 ± 2.3^{c} | 21.6 ± 16.2^{b} | 29.4 ± 17.5^{ab} | 35.7 ± 19.7^{a} | 0.04 |
| ACC 10-year ASCVD Risk (%) | 1.8 ± 1.2^{b} | 15.6 ± 11.7^{a} | 18.6 ± 7.5^{a} | 25.7 ± 12.3^{a} | 0.05 |

Values with superscript (a) have higher values at p ≤ 0.05 than values with superscript (b). Quantitative data were compared using Student's t-test. Proportions were compared using Fisher's exact test.

Oxylipin concentrations decreased with greater number of obstructed arteries; six (6) of twenty-two (22) individual oxylipins significantly decreased with the number of obstructed arteries (Table 4). In Table 5, oxylipins were grouped by FA precursors (i.e., LA, ARA, EPA, DHA), oxylipin groups (i.e., MidHODE, EET, MidHETE, EpDPA, DiHDPA), enzymes involved in their synthesis (i.e., oxygenation of PUFAs by LOX followed by reduction or alternatively hydroxylation of PUFAs by CYP1B1; oxidation of PUFAs by CYP450 followed by hydroxylation of oxidized PUFAs by soluble epoxide hydrolase (sEH)), and based on enzymatic product to substrate ratio (i.e., hydroxylation of 10(11)-EpDPA to 10(11)-DiHDPA, 14(15)-EET to 14(15)-DiHET, or 19(20)-EpDPA to 19(20)-DiHDPA by sEH). Total oxylipin concentrations significantly decreased with number of obstructed arteries, specifically omega-3 FA-derived oxylipins and within those hydroxylated DHA-epoxides DiHDPAs. The primary molecular target was sEH, specifically inhibition of hydroxylation of 19(20)-EpDPA to 19(20)-DiHDPA.

TABLE 4
Plasma oxylipin concentrations of adults with obstructed
coronary arteries (≥70% stenosis) and adults of the same
age range with a low coronary artery disease (CAD) risk.

| | Low | Number of obstructed arteries | | | |
|---|---|---|---|---|---|
| | CAD Risk | One | Two | Three | Contrast |
| | (n = 23) | (n = 31) | (n = 23) | (n = 20) | CAD1/2 |
| | Median | Median | Median | Median | vs. CAD3 |
| Oxylipins (nM) | (IQR) | (IQR) | (IQR) | (IQR) | P-value |
| 12(13)-DiHOME | 8.71^{a} | 6.97^{ab} | 5.59^{bc} | 5.11^{c} | 0.07 |
| | (5.27, 11.8) | (4.76, 8.85) | (4.07, 8.45) | (4.17, 5.89) | |
| 13(S)-HODE | 29.1^{b} | 37.6^{a} | 34.1^{ab} | 31.0^{ab} | 0.64 |
| | (26.7, 33.8) | (25.5, 49.9) | (23.4, 41.7) | (24.4, 43.2) | |
| 9(S)-HODE | 19.0 | 22.3 | 20.3 | 20.5 | 0.64 |
| | (17.6, 21.6) | (16.8, 28.8) | (16.9, 25.0) | (14.9, 26.7) | |
| Leukotriene B4 | 0.19^{ab} | 0.23^{a} | 0.24^{a} | 0.18^{b} | 0.01 |
| | (0.15, 0.24) | (0..18, 0.28) | (0.16, 0.27) | (0.15, 0.21) | |
| Thromboxane B2 | 0.05 | 0.03 | 0.04 | 0.04 | 0.10 |
| | (0.03, 0.08) | (0.02, 0.05) | (0.02, 0.04) | (0.03, 0.06) | |
| 20-HETE | 9.18 | 8.60 | 9.37 | 8.43 | 0.36 |
| | (7.04, 11.2) | (7.39, 10.4) | (7.35, 10.7) | (6.98, 9.52) | |
| 14(15)-EET | 0.27^{b} | 0.34^{a} | 0.22^{b} | 0.35^{a} | 0.70 |
| | (0.18, 0.34) | (0.28, 0.45) | (0.18, 0.36) | (0.23, 0.40) | |
| 11(12)-EET | 0.25^{b} | 0.32^{a} | 0.32^{a} | 0.35^{a} | 0.35 |
| | (0.19, 0.30) | (0.26, 0.41) | (0.27, 0.37) | (0.28, 0.46) | |
| 14(15)DiHET | 0.98^{ab} | 1.12^{a} | 0.99^{ab} | 0.90^{b} | 0.08 |
| | (0.78, 1.13) | (0.93, 1.23) | (0.84, 1.17) | (0.78, 1.09) | |
| 5-HETE | 2.69^{b} | 4.63^{a} | 4.37^{a} | 3.87^{ab} | 0.38 |
| | (2.30, 3.14) | (3.37, 7.29) | (2.87, 6.86) | (2.21, 6.65) | |
| 12-HETE | 2.41^{b} | 8.14^{a} | 11.2^{a} | 7.92^{a} | 0.10 |
| | (1.13, 4.19) | (5.60, 20.8) | (6.51, 19.6) | (2.78, 12.0) | |
| 15-HETE | 1.18^{b} | 2.41^{a} | 2.39^{a} | 2.26^{a} | 0.47 |
| | (1.02, 1.53) | (1.84, 3.29) | (1.73, 3.49) | (1.47, 3.11) | |
| 8-iso PGF3a | 0.93^{a} | 0.99^{a} | 0.87^{ab} | 0.58^{b} | 0.02 |
| | (0.49, 1.58) | (0.55, 1.95) | (0.40, 2.70) | (0.37, 0.81) | |
| 17(18)-EpETE | 0.29 | 0.26 | 0.26 | 0.29 | 0.51 |
| | (0.23, 0.35) | (0.20, 0.35) | (0.21, 0.34) | (0.24, 0.33) | |
| 11(12)-DiHETE | 0.08 | 0.06 | 0.09 | 0.05 | 0.07 |
| | (0.04, 0.11) | (0.04, 0.12) | (0.06, 0.12) | (0.01, 0.09) | |
| 19(20)-EpDPA | 0.53 | 0.59 | 0.50 | 0.60 | 0.37 |
| | (0.32, 0.95) | (0.43, 0.91) | (0.40, 0.76) | (0.48, 0.83) | |
| 10(11)-EpDPA | 0.11 | 0.11 | 0.12 | 0.11 | 0.16 |
| | (0.05, 0.16) | (0.07, 0.14) | (0.09, 0.20) | (0.07, 0.15) | |
| 19(20)-DiHDPA | 2.22^{a} | 1.92^{a} | 1.69^{ab} | 0.60^{b} | 0.04 |
| | (1.40, 2.83) | (1.19, 2.76) | (1.00, 2.63) | (0.48, 0.83) | |
| 13(14)-DiHDPA | 0.14^{a} | 0.1 l^{ab} | 0.09^{ab} | 0.09^{b} | 0.39 |
| | (0.07, 0.20) | (0.07, 0.14) | (0.07, 0.19) | (0.06, 0.12) | |
| 16(17)-DiHDPA | 0.24^{ab} | 0.24^{a} | 0.19^{ab} | 0.17^{b} | 0.04 |
| | (0.15, 0.34) | (0.16, 0.28) | (0.14, 0.27) | (0.14, 0.21) | |
| 10(11)-DiHDPA | 0.09 | 0.11 | 0.09 | 0.08 | 0.06 |
| | (0.06, 0.15) | (0.08, 0.17) | (0.06, 0.18) | (0.05, 0.11) | |
| 7(8)-DiHDPA | 0.12 | 0.13 | 0.11 | 0.12 | 0.74 |
| | (0.08, 0.14) | (0.09, 0.16) | (0.09, 0.17) | (0.10, 0.18) | |

Values with superscript [a] have higher values at p ≤ 0.05 than values with superscript [b] using Kruskal-Wallis test.
DiHOME = dihydroxy-octadecenoic acid;
HODE = hydroxy-octadecadienoic acid;
HETE = hydroxy-eicosatetraenoic acid;
EET = epoxy-eicosatrienoic acid;
DiHET = dihydroxy-eicosatrienoic acid;
EpDPA = epoxy-docosapentaenoic acid;
DiHDPA = dihydroxy-docosapentaenoic acid.

TABLE 5
Plasma concentrations of oxylipin groups in adults with obstructed
coronary arteries (≥70% stenosis) and adults of the same
age with a low coronary artery disease (CAD) risk.

| | Low | Number of obstructed arteries | | | |
|---|---|---|---|---|---|
| | CAD Risk | One | Two | Three | Contrast |
| | (n = 23) | (n = 31) | (n = 23) | (n = 20) | CAD1/2 |
| | Median | Median | Median | Median | vs. CAD3 |
| Oxylipins (nM) | (IQR) | (IQR) | (IQR) | (IQR) | P-value |
| Total | 80.3^{b} | 109^{a} | 97.9^{ab} | 85.4^{b} | 0.05 |
| | (75.9, 102) | (85.8, 171) | (82.0, 116) | (74.4, 108) | |

Fatty acid precursor

| | | | | | |
|---|---|---|---|---|---|
| C18:2 derived | 47.7^{b} | 59.7^{a} | 54.1^{ab} | 55.5^{ab} | 0.64 |
| | (43.9, 54.7) | (41.7, 76.7) | (47.4, 63.0) | (45.0, 73.4) | |
| C20:4 derived | 18.5^{b} | 28.0^{a} | 28.6^{a} | 24.4^{a} | 0.11 |
| | (14.8, 21.3) | (22.0, 40.7) | (22.6, 42.8) | (18.5, 28.9) | |
| C20:5 derived | 1.35^{a} | 1.41^{a} | 1.14^{ab} | 0.94^{b} | 0.04 |
| | (0.91, 2.01) | (0.83, 2.48) | (0.76, 3.08) | (0.64, 1.24) | |
| C22:6 derived | 3.36^{a} | 3.36^{ab} | 3.14^{ab} | 2.59^{b} | 0.18 |
| | (2.26, 5.06) | (2.16, 4.25) | (2.01, 4.03) | (2.20, 3.19) | |

Oxylipin group

| | | | | | |
|---|---|---|---|---|---|
| MidHODE | 47.7^{b} | 59.7^{a} | 54.1^{ab} | 51.2^{ab} | 0.64 |
| | (44.7, 54.7) | (41.7, 76.7) | (47.4, 63.0) | (40.1, 68.7) | |
| EET | 1.45^{b} | 1.77^{a} | 1.63^{ab} | 1.51^{ab} | 0.47 |
| | (1.28, 1.68) | (1.45, 2.16) | (1.32, 1.87) | (1.39, 1.89) | |
| MidHETE | 6.34^{b} | 17.1^{a} | 19.0^{a} | 13.2^{a} | 0.11 |
| | (5.06, 9.50) | (12.1, 27.0) | (10.6, 32.4) | (8.07, 19.8) | |
| EpDPA | 0.67 | 0.75 | 0.66 | 0.74 | 0.72 |
| | (0.36, 1.06) | (0.51, 1.13) | (0.50, 0.94) | (0.57, 0.99) | |
| DiHDPA | 2.84^{a} | 2.50^{a} | 2.34^{ab} | 1.89^{b} | 0.04 |
| | (1.89, 3.70) | (1.63, 3.45) | (1.37, 3.28) | (1.37, 2.33) | |

Enzyme products

| | | | | | |
|---|---|---|---|---|---|
| LOX/CYP1B1 | 55.8^{b} | 78.9^{a} | 79.1^{a} | 68.8^{ab} | 0.27 |
| products | (53.0, 68.0) | (60.7, 114) | (59.3, 92.3) | (55.4, 89.4) | |
| LOX12-15 | 32.9^{b} | 47.9^{a} | 53.8^{a} | 43.5^{a} | 0.26 |
| products | (19.7, 40.4) | (37.2, 72.2) | (31.5, 62.5) | (31.6, 55.1) | |
| LOX5 products | 21.9^{b} | 27.2^{a} | 26.5^{ab} | 25.1^{ab} | 0.55 |
| | (20.1, 25.6) | (21.0, 37.3) | (19.9, 30.2) | (18.4, 31.4) | |
| CYP epoxides | 1.44 | 1.70 | 1.56 | 1.94 | 0.86 |
| | (1.19, 1.90) | (1.41, 2.19) | (1.22, 2.14) | (1.32, 2.11) | |
| Hydroxylated CYP | 11.8^{a} | 10.0^{ab} | 9.50^{bc} | 7.77^{c} | 0.008 |
| epoxides | (8.61, 20.8) | (8.02, 14.1) | (7.35, 11.9) | (6.40, 9.21) | |

CYP450 product to substrate ratios

| | | | | | |
|---|---|---|---|---|---|
| 10(11)-DiHDPA/ | 0.90^{a} | 0.81^{b} | 0.79^{b} | 0.71^{b} | 0.21 |
| 10(11)-EpDPA | (0.78, 1.30) | (0.63, 0.93) | (0.59, 1.04) | (0.52, 0.92) | |
| 14(15)-DiHET/ | 4.03^{a} | 2.88^{b} | 3.76^{a} | 2.56^{b} | 0.08 |
| 14(15)-EET | (2.91, 5.34) | (2.39, 4.13) | (2.63, 5.37) | (2.29, 4.17) | |
| 19(20)-DiHDPA/ | 4.26^{a} | 2.97^{a} | 2.95^{a} | 1.91^{b} | 0.002 |
| 19(20 EpEPA | (2.48, 6.55) | (2.19, 3.92) | (2.13, 4.77) | (1.58, 2.98) | |

Values with superscript [a] have higher values at p ≤ 0.05 than values with superscript [b] using Kruskal-Wallis test.
HODE = hydroxy-octadecadienoic acid;
HETE = hydroxy-eicosatetraenoic acid;
EET = epoxy-eicosatrienoic acid;
DiHET = dihydroxy-eicosatrienoic acid;
EpDPA = epoxy-docosapentaenoic acid;
DiHDPA = dihydroxy-docosapentaenoic acid;
LOX = lipoxygenase;
CYP = cytochrome P450.
CABG = coronary artery bypass grafting.

Low CAD risk adults had lower total oxylipin concentrations than adults with CAD. Specifically, omega-6 FA-derived oxylipins and within those MidHETEs (Tables 4, 5). These include three (3) individual oxylipins that were significantly lower than in each CAD group: 11(12)-EET, 12-HETE, and 15-HETE. The primary molecular targets were LOX 12-15 enzymes or CYP1B1, which are involved in oxygenation of omega-6 FA. Less hydroxylation of 10(11)-EpDPA to 10(11)-DiHDPA was also observed. Concentrations of LA-derived 12(13)-DiHOME and DHA-derived DiHDPAs, specifically 19(20)-DiHDPA and 16(17)-DiHDPA, decreased gradually from adults with low CAD risk to those with three (3) diseased arteries.

### Diagnostic Efficacy of Oxylipins

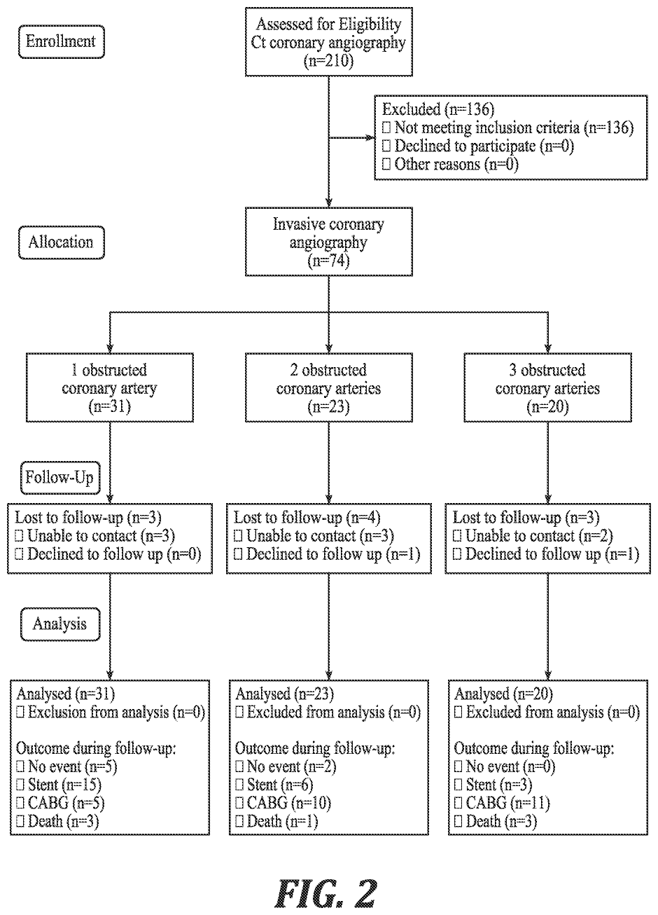

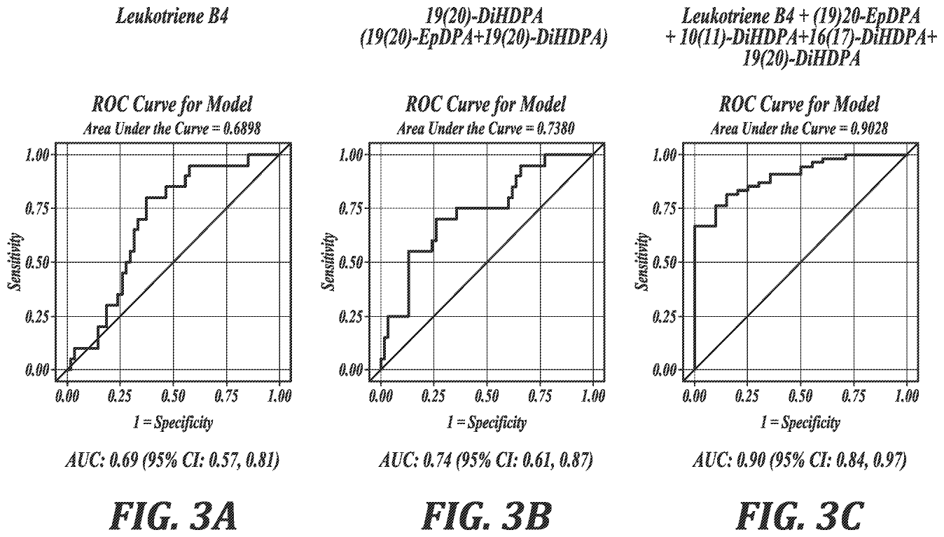

Changes in plasma oxylipin concentrations were primarily between 2 and 3 diseased vessels. Among individual oxylipins, ARA-derived leukotriene B4 could best diagnose three (3) obstructed arteries (AUC: 0.69; 95% CI: 0.57 to 0.81; P=0.003) (FIG. 3A). Leukotriene B4 concentrations ≤0.21 nM diagnosed three (3) obstructed arteries in 80% of CAD3 adults less obstructed arteries in 65% CAD1 adults, 61% CAD2 adults, and 43% adults with low CAD risk.

Significant AUC values were also observed for EPA-derived 8-iso PGF3a (AUC: 0.67; 95% CI: 0.54 to 0.80; P=0.009), three DHA-derived DiHDPA 19(20)-DiHDPA (AUC: 0.66; 95% CI: 0.54 to 0.78; P=0.01), 16(17)-DiHDPA (AUC: 0.65; 95% CI: 0.52 to 0.79; P=0.02), 10(11)-DiHDPA (AUC: 0.64; 95% CI: 0.51 to 0.78; P=0.04), and LA-derived 12(13)-DiHOME (AUC: 0.64; 95% CI: 0.51 to 0.77; P=0.04).

Among oxylipin groups and ratios, three (3) obstructed arteries was best diagnosed by the 19(20)-DiHDPA fraction of the sum of 19(20)-EpDPA and 19(20)-DiHDPA (AUC: 0.74; 95% CI: 0.61 to 0.87; P=0.0003; FIG. 3B). A fraction of <72% diagnosed three (3) obstructed arteries in 70% of CAD3 adults and less obstructed arteries in 74% CAD1 adults, 70% CAD2 adults, and 78% adults with low CAD risk. Adding 8-iso PGF3a to the fraction improved diagnosis of three (3) obstructed arteries to 80% but decreased diagnosis of less obstructed arteries to 60% in CAD2 adults. An oxylipin panel of leukotriene B4, 19(20)-EpDPA, 19(20)-DiHDPA, 13(14)-DiHDPA, and 10(11)-DiHDPA diagnosed three (3) obstructed arteries in all CAD3 adults and less obstructed arteries in 70% CAD1 and CAD2 adults (AUC: 0.90; 95% CI: 0.84 to 0.97; P<0.0001; FIG. 3C). The oxylipin panel improved (P=0.02) diagnosis of three (3) obstructed arteries compared to the 10-year Framingham general CVD risk score (AUC: 0.68; 95% CI: 0.52 to 0.83; P=0.02).

Prediction of Outcomes in Adults with Obstructed Coronary Arteries

Adults with CAD were followed up until November 2019 for a median of 5 years (range: 25 to 84 months) and adverse events were recorded (i.e., coronary stent placement; CABG surgery; death). Ten participants (3 women and 7 men; median age: 61 years; range: 51 to 81 years) were lost to follow up (FIG. 2). Given the degree of obstruction, 52 of 64 adults with CAD underwent open-heart surgery within 3 months of the angiogram (CAD1: 19 of 28; CAD2: 16 of 19; CAD3: 17 of 17): 28 had a CABG surgery (CAD1: 5 of 19; CAD2: 10 of 16; CAD3: 13 of 17) and 26 had a coronary stent placement (CAD1: 16; CAD2: 6; CAD3: 4). Adults with multiple diseased coronary arteries were more likely to undergo open-heart surgery and receive a CABG. Of the remaining 12 adults with CAD, seven had no further event, two CAD1 adults received a coronary stent during follow up, and three died (CAD1: 2; CAD2: 1). In addition, 4 CAD adults that had undergone open-heart surgery within 3 months (2 stents and 2 CABG) died during follow-up (CAD1: 1; CAD2: 0; CAD3: 3). Survival was not linked to number of diseased coronary arteries.

Table 6 lists selected demographic and clinical characteristics of adults with obstructed coronary arteries (≥70% stenosis) based on outcomes during follow-up. Survival was linked to lower systolic blood pressure or being a male, whereas survival without CABG was linked to higher plasma triacylglycerol concentrations. Unfavorable outcomes were linked to elevated oxylipin concentrations (Table 7), specifically omega-6 FA-derived oxylipins and within those LA-derived MidHODEs and ARA-derived MidHETEs (Table 8). Concentrations of LA-derived 9(S)-HODE and 13(S)-HODE and ARA-derived thromoboxane B2, 5-HETE and 15-HETE increased gradually from stent placement to CABG to death. In contrast, EPA-derived 8-iso PGF3a were lower with unfavorable outcomes. The primary molecular target were PUFA-oxygenating LOX enzymes or PUFA-hydroxylating CYP1B1 enzyme.

TABLE 6
Demographic and clinical characteristics of adults with obstructed coronary
arteries (≥70% stenosis) satisfied by outcome during 5-year follow up.

| | Outcome during follow-up | | | | | | | | Contrasts | | |
|---|---|---|---|---|---|---|---|---|---|---|---|
| | No Event | | Stent | | CABG | | Death | | No Event | CABG/Death | Death |
| | (n = 7) | | (n = 24) | | (n = 26) | | (n = 7) | | vs. Others | vs. Others | vs. Others |
| Characteristics | Mean ± STD | | Mean ± STD | | Mean ± STD | | Mean ± STD | | P-value | P-value | P-value |
| Age (yr) | 65 ± 8 | | 68 ± 8 | | 67 ± 10 | | 60 ± 17 | | 0.71 | 0.52 | 0.31 |
| Male, n (%) | 3 | (43)^{bc} | 19 | (79)^{ab} | 22 | (85)^{a} | 2 | (29)^{c} | 0.08 | 1 | 0.02 |
| BMI (kg/m²) | 30.2 ± 8.5 | | 29.9 ± 8.8 | | 28.3 ± 3.3 | | 29.9 ± 5.6 | | 0.70 | 0.46 | 0.78 |
| Overweight, n (%) | 1 | (14)^{b} | 9 | (38)^{ab} | 16 | (62)^{a} | 4 | (57)^{ab} | 0.11 | 0.03 | 0.70 |
| Obese, n (%) | 3 | (43) | 9 | (38) | 7 | (27) | 2 | (29) | 0.67 | 0.43 | 1 |

Blood Pressure (mm Hg)

| | | | | | | | |
|---|---|---|---|---|---|---|---|
| Systolic | 130 ± 16^{ab} | 130 ± 16^{b} | 130 ± 19^{ab} | 147 ± 15^{a} | 0.81 | 0.42 | 0.02 |
| Diastolic | 65 ± 12 | 72 ± 10 | 70 ± 11 | 67 ± 8 | 0.20 | 0.63 | 0.42 |

Plasma:

| Triacylglycerol (mg/dL) | 197 ± 131 | | 167 ± 100 | | 123 ± 59 | | 89 ± 42 | | 0.29 | 0.02 | 0.13 |
|---|---|---|---|---|---|---|---|---|---|---|---|
| Total Cholesterol (mg/dL) | 174 ± 44 | | 187 ± 50 | | 162 ± 42 | | 153 ± 46 | | 0.91 | 0.06 | 0.29 |
| HDL-Cholesterol (mg/dL) | 48 ± 14 | | 49 ± 12 | | 44 ± 18 | | 53 ± 14 | | 0.94 | 0.51 | 0.39 |
| LDL-Cholesterol (mg/dL) | 92 ± 35 | | 104 ± 37 | | 94 ± 41 | | 73 ± 39 | | 0.79 | 0.29 | 0.17 |
| Hbalc (mmol/mol) | 5.9 ± 0.8 | | 6.9 ± 0.4 | | 6.2 ± 1.3 | | 6.5 ± 2.5 | | 0.61 | 0.74 | 0.95 |
| Medication, n (%) | 7 | (100) | 23 | (96) | 25 | (96) | 7 | (100) | 1 | 1 | 1 |
| Blood Pressure, Total | 6 | (86) | 22 | (92) | 22 | (85) | 6 | (86) | 1 | 0.71 | 1 |
| ACE Inhibitor | 2 | (29) | 10 | (42) | 6 | (23) | 2 | (29) | 1 | 0.28 | 1 |
| AR Blocker | 2 | (29) | 4 | (17) | 3 | (12) | 2 | (29) | 0.59 | 0.75 | 0.59 |
| Beta Blocker | 3 | (43)^{b} | 20 | (83)^{a} | 17 | (65)^{ab} | 4 | (57)^{ab} | 0.19 | 0.43 | 0.67 |
| Calcium Channel Blocker | 3 | (43) | 4 | (17) | 6 | (23) | 1 | (14) | 0.17 | 1 | 1 |
| Diabetes, Total | 2 | (29) | 5 | (21) | 6 | (23) | 3 | (43) | 1 | 0.78 | 0.35 |
| Oral Hyperglycemia | 2 | (29) | 3 | (13) | 3 | (12) | 1 | (14) | 0.25 | 0.73 | 1 |

| | | | | | | | | | | |
|---|---|---|---|---|---|---|---|---|---|---|
| Insulin | 0 | 2 | (8) | 3 | (12) | 2 | (29) | 1 | 0.43 | 0.17 |

Hyperlipidemia, Total

| | | | | | | | | | | | |
|---|---|---|---|---|---|---|---|---|---|---|---|
| Statin | 6 | (86) | 19 | (79) | 20 | (77) | 5 | (71) | 1 | 0.77 | 0.64 |
| Aspirin | 6 | (86) | 20 | (83) | 21 | (81) | 5 | (71) | 1 | 0.75 | 0.61 |

CAD Risk factors:
Tobacco Use, n (%)

| Former | 0 | | 9 | (40) | 8 | (31) | 1 | (14) | 0.18 | 1 | 0.12 |
|---|---|---|---|---|---|---|---|---|---|---|---|
| Active | 1 | (14) | 2 | (8) | 4 | (15) | 3 | (43) | 1 | 0.30 | 0.07 |

History of, n (%):

| | | | | | | | | | | | |
|---|---|---|---|---|---|---|---|---|---|---|---|
| Hypertension | 6 | (86) | 22 | (92) | 22 | (85) | 7 | (100) | 0.57 | 1 | 1 |
| Diabetes | 3 | (43) | 6 | (25) | 8 | (31) | 4 | (57) | 0.67 | 0.60 | 0.20 |
| Hypercholesterolemia | 7 | (100) | 24 | (100) | 23 | (88) | 7 | (100) | 1 | 0.49 | 1 |
| CVD in Family | 4 | (57) | 12 | (50) | 13 | (50) | 3 | (43) | 1 | 1 | 1 |

| | | | | | | | |
|---|---|---|---|---|---|---|---|
| Total Risk (1-5) | 3.0 ± 0.8^{ab} | 2.8 ± 0.7^{b} | 2.7 ± 1.0^{ab} | 3.4 ± 1.0^{a} | 0.60 | 0.85 | 0.06 |
| Framingham 10-yr CVD Risk, % | 23.0 ± 18.8 | 28.0 ± 17.9 | 30.1 ± 18.3 | 28.4 ± 22.2 | 0.41 | 0.53 | 0.98 |
| ACC 10-year ASCVD Risk, % | 17.5 ± 11.9 | 18.9 ± 10.1 | 20.9 ± 13.8 | 9.0 | 0.77 | 0.77 | ND |

Values with superscript [a] have higher values at p ≤ 0.05 than values with superscript [b]. Quantitative data were compared using Student's t-test. Proportions were compared using Fisher's exact test.
CABG = coronary artery bypass grafting.
ACE = angiotension converting enzyme;
AR = angiotension receptor;
ACC = American College of Cardiology;
ASCVD = Atherosclerotic Cardiovascular Disease

TABLE 7
Plasma oxylipin concentrations of adults with obstructed coronary arteries
(≥70% stenosis) stratified by outcome during 5-year folow up.

| | Outcome during follow-up | | | | | | |
|---|---|---|---|---|---|---|---|
| | No Event | Stent | CABG | Death | Contrasts | | |
| | (n = 7) | (n = 24) | (n = 26) | (n = 7) | No Event | CABG/Death | Death |
| | Median | Median | Median | Median | vs. Others | vs. Others | vs. Others |
| Oxylipins (nM) | (IQR) | (IQR) | (IQR) | (IQR) | P-value | P-value | P-value |
| 12(13)-DiHOME | 5.05 | 5.71 | 5.47 | 5.69 | 0.38 | 0.66 | 0.82 |
| | (3.81, 6.68) | (4.25, 7.64) | (4.29, 8.56) | (5.09, 6.93) | | | |
| 13(S)-HODE | 33.9^{cb} | 27.4^{b} | 34.1^{b} | 46.2^{a} | 0.71 | 0.04 | 0.007 |
| | (18.5, 37.8) | (17.6, 40.2) | (25.2, 42.2) | (42.6, 58.9) | | | |
| 9(S)-HODE | 19.4^{cb} | 20.0^{b} | 20.9^{b} | 30.6^{a} | 0.65 | 0.03 | 0.01 |
| | (12.2, 25.8) | (11.5, 22.4) | (17.1, 25.8) | (21.9, 56.9) | | | |
| Leukotriene B4 | 0.22 | 0.24 | 0.22 | 0.19 | 0.47 | 0.36 | 0.14 |
| | (0.15, 0.24) | (0.17, 0.28) | (0.17, 0.25) | (0.16, 0.22) | | | |
| Thromboxane B2 | 0.04^{ab} | 0.03^{b} | 0.04^{a} | 0.04^{a} | 0.89 | 0.04 | 0.08 |
| | (0.02, 0.11) | (0.02, 0.04) | (0.02, 0.06) | (0.04, 0.12) | | | |
| 20-HETE | 8.03 | 8.57 | 8.62 | 8.63 | 0.54 | 0.44 | 0.58 |
| | (7.75, 9.93) | (6.88, 10.2) | (8.11, 10.4) | (5.66, 9.20) | | | |
| 14(15)-EET | 0.31 | 0.28 | 0.36 | 0.29 | 0.89 | 0.20 | 0.67 |
| | (0.22, 0.41) | (0.20, 0.38) | (0.22, 0.46) | (0.18, 0.38) | | | |
| 11(12)-EET | 0.35^{ab} | 0.29^{b} | 0.36^{a} | 0.32^{ab} | 0.72 | 0.08 | 0.92 |
| | (0.27, 0.41) | (0.23, 0.38) | (0.22, 0.46) | (0.24, 0.39) | | | |
| 14(15)-DiHET | 1.04 | 0.97 | 1.05 | 1.03 | 0.79 | 0.38 | 0.97 |
| | (0.87, 1.23) | (0.74, 1.13) | (0.89, 1.19) | (0.86, 1.18) | | | |
| 5-HETE | 5.87^{a} | 3.68^{b} | 4.50^{ab} | 5.00^{a} | 0.07 | 0.31 | 0.04 |
| | (4.12, 11.08) | (2.28, 5.00) | (2.89, 5.98) | (4.43, 8.63) | | | |
| 12-HETE | 8.14 | 7.00 | 10.9 | 11.1 | 0.97 | 0.09 | 0.38 |
| | (5.42, 11.2) | (4.60, 10.0) | (5.54, 20.8) | (5.48, 25.5) | | | |
| 15-HETE | 2.92^{ab} | 1.86^{b} | 2.52^{a} | 2.44^{a} | 0.51 | 0.03 | 0.27 |
| | (1.84, 2.97) | (1.48, 2.34) | (2.05, 3.19) | (2.13, 4.17) | | | |
| 8-iso PGF3a | 0.60^{b} | 1.48^{a} | 0.64^{ab} | 0.39^{b} | 0.28 | 0.08 | 0.06 |
| | (0.29, 0.99) | (0.60, 14.6) | (0.43, 1.95) | (0.34, 0.72) | | | |
| 17(18)EpETE | 0.22 | 0.28 | 0.29 | 0.31 | 0.42 | 0.68 | 0.34 |
| | (0.19, 0.33) | (0.22, 0.35) | (0.19, 0.35) | (0.24, 0.39) | | | |
| 11(12)-DiHETE | 0.06 | 0.05 | 0.08 | 0.06 | 0.67 | 0.81 | 0.57 |
| | (0.04, 0.19) | (0.03, 0.13) | (0.04, 0.12) | (0.04, 0.09) | | | |
| 19(20)-EpDPA | 0.70 | 0.51 | 0.64 | 0.49 | 0.33 | 0.54 | 0.38 |
| | (0.47, 1.04) | (0.28, 0.76) | (0.46, 0.83) | (0.40-0.56) | | | |
| 10(11)-EpDPA | 0.12 | 0.10 | 0.13 | 0.09 | 0.35 | 0.93 | 0.60 |
| | (0.10, 0.38) | (0.06, 0.19) | (0.06, 0.20) | (0.08, 0.11) | | | |
| 19(20)-DiHDPA | 1.61 | 1.63 | 1.44 | 1.78 | 0.63 | 0.64 | 0.45 |
| | (1.26, 2.81) | (0.91, 2.36) | (0.97, 2.18) | (1.19, 2.73) | | | |
| 13(14)-DiHDPA | 0.07 | 0.10 | 0.11 | 0.08 | 0.97 | 0.91 | 0.20 |
| | (0.06, 0.21) | (0.06, 0.15) | (0.08, 0.15) | (0.04, 0.12) | | | |
| 16(17)-DiHDPA | 0.20 | 0.20 | 0.18 | 0.20 | 0.43 | 0.43 | 0.99 |
| | (0.15, 0.41) | (0.13, 0.28) | (0.14, 0.24) | (0.15, 0.24) | | | |
| 10(11)-DiHDPA | 0.11 | 0.09 | 0.09 | 0.08 | 0.58 | 0.79 | 0.48 |
| | (0.04, 0.33) | (0.05, 0.13) | (0.06, 0.16) | (0.06, 0.11) | | | |
| 7(8)-DiHDPA | 0.15 | 0.12 | 0.12 | 0.14 | 0.48 | 0.82 | 0.67 |
| | (0.08, 0.20) | (0.08, 0.15) | (0.09, 0.17) | (0.11, 0.18) | | | |

Values with superscript [a] have higher values at p ≤ 0.05 than values with superscript [b] using Kruskal-Wallis test.
CABG = coronary artery bypass grafting.
DiHOME = dihydroxy-octadecenoic acid;
HODE = hydroxy-octadecadienoic acid;
HETE = hydroxy-eicosatetraenoic acid;
EET = epoxy-eicosatrienoic acid;
DiHET = dihydroxy-eicosatrienoic acid;
EpDPA = epoxy-docosapentaenoic acid;
DiHDPA = dihydroxy-docosapentaenoic acid

TABLE 8
Plasma concentrations of oxylipin groups in adults with obstructed coronary
arteries (≥70% stenosis) stratified by outcome during 5-year follow up.

| | Outcomes during follow-up | | | | | | |
|---|---|---|---|---|---|---|---|
| | No Event | Stent | CABG | Death | Contrasts | | |
| | (n = 7) | (n = 24) | (n = 26) | (n = 7) | No Event | CABG/Death | Death |
| | Median | Median | Median | Median | vs. Others | vs. Others | vs. Others |
| Oxylipins (nM) | (IQR) | (IQR) | (IQR) | (IQR) | P-value | P-value | P-value |
| Total | 97.8^{ab} | 86.4^{b} | 103^{ab} | 119^{a} | 0.71 | 0.16 | 0.05 |
| | (62.2, 112) | (69.4, 132) | (81.4, 116) | (97.4, 180) | | | |

Fatty acid precursor

| | | | | | | | |
|---|---|---|---|---|---|---|---|
| C18:2 derived | 59.7^{b} | 47.6^{b} | 54.9^{b} | 76.7^{a} | 0.76 | 0.04 | 0.004 |
| | (30.7, 62.4) | (28.9, 62.5) | (45.9, 65.8) | (64.5, 135) | | | |
| C20:4 derived | 28.0^{ab} | 23.3^{b} | 28.4^{a} | 29.0^{a} | 0.51 | 0.03 | 0.19 |
| | (24.9, 35.6) | (18.3, 30.5) | (23.6, 41.1) | (27.3, 44.9) | | | |
| C20:5 derived | 0.87^{ab} | 1.95^{a} | 0.97^{ab} | 0.86^{b} | 0.31 | 0.13 | 0.17 |
| | (0.67, 1.37) | (0.87, 14.9) | (0.74, 2.48) | (0.66, 1.07) | | | |
| C22:6 derived | 3.02 | 2.95 | 2.58 | 2.96 | 0.58 | 0.80 | 0.84 |
| | (2.25, 5.86) | (1.48, 4.21) | (2.13, 3.93) | (2.13, 3.66) | | | |

Oxylipin group

| | | | | | | | |
|---|---|---|---|---|---|---|---|
| MidHODE | 59.7^{b} | 47.6^{b} | 54.9^{b} | 76.7^{a} | 0.76 | 0.04 | 0.004 |
| | (30.7, 62.4) | (28.9, 62.5) | (45.9, 65.8) | (64.5, 135) | | | |
| EET | 1.71 | 1.51 | 1.75 | 1.46 | 0.84 | 0.20 | 0.76 |
| | (1.45, 1.80) | (1.37, 1.83) | (1.44, 2.25) | (1.32, 1.99) | | | |
| MidHETE | 17.4^{a} | 12.9^{b} | 18.0^{ab} | 17.9^{a} | 0.36 | 0.08 | 0.17 |
| | (15.2, 21.4) | (10.1, 18.4) | (11.5, 30.8) | (17.1, 34.1) | | | |
| EpDPA | 0.82 | 0.63 | 0.77 | 0.56 | 0.32 | 0.59 | 0.35 |
| | (0.53, 1.42) | (0.33, 0.92) | (0.57, 0.97) | (0.50, 0.75) | | | |
| DiHDPA | 2.07 | 2.13 | 1.89 | 2.26 | 0.64 | 0.67 | 0.61 |
| | (1.72, 4.05) | (1.24, 3.01) | (1.35, 2.83) | (1.58, 3.27) | | | |

Enzyme products

| | | | | | | | |
|---|---|---|---|---|---|---|---|
| LOX/CYP1B1 | 77.7^{ab} | 62.6^{b} | 73.6^{b} | 96.4^{a} | 0.94 | 0.03 | 0.01 |
| products | (47.2, 89.7) | (41.9, 89.6) | (61.7, 92.3) | (87.0, 154) | | | |
| LOX12-15 | 46.2^{bc} | 37.4^{c} | 44.9^{b} | 57.1^{a} | 0.85 | 0.02 | 0.02 |
| products | (30.3, 48.9) | (24.9, 58.4) | (38.0, 62.5) | (54.7, 72.2) | | | |
| LOX5 | 26.7^{ab} | 23.9^{b} | 25.3^{b} | 37.1^{a} | 0.92 | 0.07 | 0.007 |
| products | (18.3, 37.3) | (16.0, 28.7) | (21.4, 30.7) | (28.7, 64.8) | | | |
| CYP epoxides | 1.97 | 1.57 | 1.83 | 1.56 | 0.37 | 0.46 | 0.58 |
| | (1.41, 2.19) | (1.07, 1.95) | (1.34, 2.17) | (1.27, 1.97) | | | |
| Hydroxylated CYP | 10.0 | 9.19 | 9.22 | 9.05 | 0.89 | 0.91 | 0.94 |
| epoxides | (6.94, 10.4) | (7.50, 10.8) | (7.29, 12.0) | (7.96, 9.63) | | | |

CYP450 product to substrate ratios

| | | | | | | | |
|---|---|---|---|---|---|---|---|
| 10(11)-diHDPA/ | 0.79 | 0.79 | 0.79 | 0.78 | 0.89 | 0.88 | 0.96 |
| 10(11)-EpDPA | (0.64, 0.94) | (0.60, 0.96) | (0.57, 1.04) | (0.58, 0.93) | | | |
| 14(15)-DiHet/ | 2.83 | 3.31 | 2.79 | 2.96 | 0.99 | 0.66 | 0.71 |
| 14(15)-EET | (2.39, 5.27) | (2.54, 4.39) | (2.31, 4.17) | (2.51, 5.95) | | | |
| 19(20)-DiHDPA/ | 2.85^{ab} | 3.83^{a} | 2.57^{b} | 3.41^{ab} | 0.22 | 0.25 | 0.23 |
| 19(20)-EpDPA | (1.61, 3.11) | (2.13, 4.39) | (1.70, 3.31) | (2.69, 4.86) | | | |

Values with superscript [a] have higher values at p ≤ 0.05 than values with superscript [b] using Kruskal-Wallis test.
CABG = coronary artery bypass grafting.
HODE = hydroxy-octadecadienoic acid;
HETE = hydroxy-eicosatetraenoic acid;
EET = epoxy-eicosatrienoic acid;
DiHET = dihydroxy-eicosatrienoic acid;
EpDPA = epoxy-docosapentaenoic acid;
DiHDPA = dihydroxy-docosapentaenoic acid;
LOX = lipoxygenase;
CYP = cytochrome P450.

### Predictive Efficacy of Oxylipins

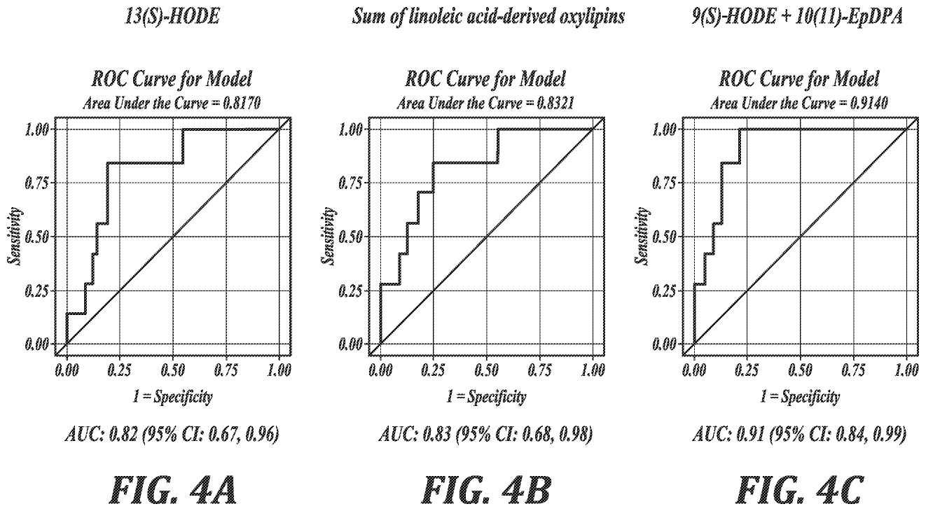

Among individual oxylipins, survival was predicted best by LA-derived 13(S)-HODE (AUC: 0.82; 95% CI: 0.67 to 0.96; P<0.0001); concentrations of 13(S)-HODE>42.5 nM predicted mortality in 86% non-surviving adults with CAD and predicted survival in 81% surviving adults with CAD and 91% Astoria cohort adults (FIG. 4A).

Adding 10(11)-EpDPA concentrations <0.20 nM for classification, improved survival prediction to 91% surviving adults with CAD and 96% Astoria cohort adults (AUC: 0.90; 95% CI: 0.81 to 0.99; P<0.0001). The two-oxylipin panel improved (P=0.02) survival prediction compared to the 10-year Framingham general CVD risk score (AUC: 0.49; 95% CI: 0.16 to 0.83; P=0.97).

The four-remaining individual oxylipins that could significantly predict survival were ordered by p-value: EPA-derived 9(S)-HODE (AUC: 0.79; 95% CI: 0.62 to 0.96; P=0.0007), ARA-derived 5-HETE (AUC: 0.73; 95% CI: 0.58 to 0.89; P=0.01), EPA-derived 8-iso PGF3a (AUC: 0.72; 95% CI: 0.54 to 0.89; P=0.02), and ARA-derived thromboxane B2 (AUC: 0.72; 95% CI: 0.54 to 0.89; P=0.03). The best single predictor for survival was the sum of LA-derived oxylipins (AUC: 0.83; 95% CI: 0.68 to 0.98; P<0.0001; FIG. 4B). The targeted AUC value of at least 0.90 was achieved with a two-oxylipin panel of 9(S)-HODE and 10(11)-EpDPA (AUC: 0.91; 95% CI: 0.84 to 0.99; P<0.0001; FIG. 4C).

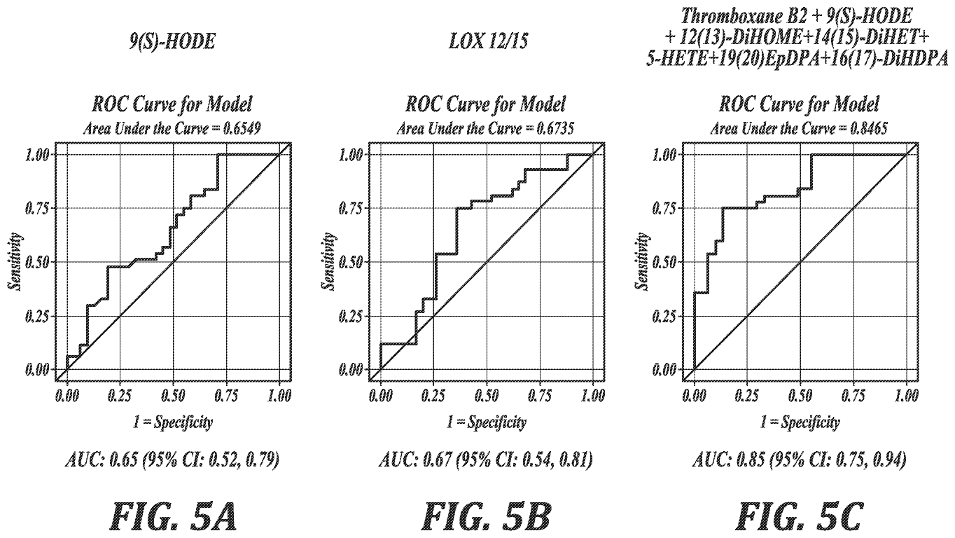

Among individual oxylipins, survival without requiring CABG was best predicted by LA-derived 9(S)-HODE (AUC: 0.65; 95% CI: 0.52 to 0.79; P=0.03; FIG. 5A). The two-remaining individual oxylipins that could significantly predict survival without requiring CABG were ordered by p-value: ARA-derived 15-HETE (AUC: 0.65; 95% CI: 0.52 to 0.79; P=0.03) and ARA-derived thromboxane B2 (AUC: 0.65; 95% CI: 0.51 to 0.79; P=0.03). The best single predictor was the sum of LOX12/15-epoxygenated oxylipins (AUC: 0.67; 95% CI: 0.54 to 0.81; P=0.01; FIG. 5B). The targeted AUC value of ≥0.85 was achieved with a linear combination of 9(S)-HODE, 5-HETE, 14(15)-DiHET, thromboxane B2, 19(20)-EPDPA, and 16(17)-DiHDPA (AUC: 0.85; 95% CI: 0.75 to 0.94; P=0.0001; FIG. 5C). The oxylipin panel improved predictive efficacy (P=0.004) compared with the 10-year Framingham general CVD risk score (AUC: 0.55; 95% CI: 0.40 to 0.71; P=0.51).

The only single oxylipin that could significantly predict CAD adults without follow-up events was ARA-derived 5-HETE (AUC: 0.71; 95% CI: 0.52 to 0.91; P=0.03). In general, patients without follow-up events had oxylipin values similar to patients who died during follow up or had a surgery for a full blockage. The 10-year Framingham general CVD risk score had an AUC of 0.62 (95% CI: 0.38 to 0.87; P=0.33).

In order to achieve a representative coverage of LA-, ARA-, EPA- and DHA-derived oxylipins and the enzymatic and non-enzymatic pathways involved in their production, a library with standards of 39 oxylipins was analyzed (Table 2). All 39 oxylipins were detected in one 22-min run. Of the 39 oxylipins, 24 were consistently above the LOD, and 22 oxylipins were consistently above the LOQ. The library included (i) 4 LA-derived oxylipins (2 each from CYP450 and LOX pathways), of which 3 (CYP450: 12(13)-DiHOME, LOX: 9(S) HODE, 13(S) HODE) were above the LOQ; (ii) 14 ARA-derived oxylipins (5 from COX, 5 from CYP450, and 4 from LOX pathways), of which 9 (COX: thromboxane B2; CYP450: 11(12)-EET, 14(15)-EET, 20-HETE, 14(15)-DiHET; LOX: 5-HETE, 12-HETE, 15-HETE, and leukotriene B4) were above the LOQ; (iii) 10 EPA-derived oxylipins (1 from COX, 8 from CYP450, and 1 from ROS pathways), of which 3 (CYP450: 11(12)-DiHETE, 17(18)-EpETE; ROS: 8-iso PGF3a) were above the LOQ; (iv) 11 DHA-derived oxylipins (10 from CYP450 and 1 from LOX pathways) of which 7 (CYP450: 10(11)-EpDPA, 19(20) EpDPA, 7(8)-DiHDPA, 10(11)-DiHDPA, 13(14)-DiHDPA, 16(17)-DiHDPA, 19(20)-DiHDPA) were above the LOQ.

Elevated concentrations of ARA-derived oxylipins were uncommon in -CAD participants (Table 5). The three best classifying oxylipins, 15-HETE (ROC: 0.86; 95% CI: 0.79 to 0.93; P<0.0001), 12-HETE (ROC: 0.79; 95 CI: 0.69 to 0.88; P<0.0001), and 5-HETE (ROC: 0.75; 95 CI: 0.66 to 0.84; P<0.0001), were all synthesized by hydroxylation of ARA by LOX enzymes. Plasma concentrations of 15-HETE<2.01 nM ruled out CAD with 97% sensitivity (36 of 37 Control participants) and 65% specificity (48 of 74+CAD participants; FIG. 2A). The specificity was increased to 78% (58 of 74+CAD participants) by combining 15-HETE<2.01 nM with 12(13)-DiHOME≥3.99 nM without losing sensitivity. The ROC value for the combination of 15-HETE and 12(13)-DiHOME reached close to our target goal of an ROC of 0.90 with 0.89 (95 CI: 0.82 to 0.95; P<0.0001; FIG. 2B). Additional oxylipins did not significantly improve CAD classification.

As a side note, 12(13)-DiHOME (ROC: 0.67; 95 CI: 0.57 to 0.78; P=0.001), which is formed by epoxygenation of LA by CYP450 enzymes, and thromboxane B2 (ROC: 0.64; 95 CI: 0.53 to 0.75; P=0.02), which is formed from ARA by COX enzymes, were the only oxylipins, the concentrations of which were higher in the absence of confirmed obstructive CAD. Furthermore, it was uncommon for -CAD participants to have elevated concentrations of ARA-derived CYP450-epoxygenated EETs 11(12)-EET (ROC: 0.72; 95 CI: 0.63 to 0.82; P<0.0001) and 14(15)-EET (ROC: 0.63; 95 CI: 0.53 to 0.74; P=0.01).

Whereas ARA-derived 15-HETE, 12-HETE, and 11(12)-EET concentrations remained elevated in all participants with confirmed obstructed arteries, the concentrations of 6 oxylipins decreased with number of obstructed coronary arteries (Table 5). Of greatest importance, is the trend observed for 12(13)-DiHOME, which was the only oxylipin, which consistently decreased from 0 to 3 obstructed coronary arteries. Using the combination of 15-HETE<2.01 nM and 12(13)-DiHOME≥7.0 nM, resulted in the correct classification of all 20 CAD3 patients, 21 of 23 CAD2 patients, and 26 of 31 CAD1 patients.

Four oxylipins gradually decreased from 1 to 3 obstructed arteries: EPA-derived 8-iso PGF3a and the DHA-derived 19(20)-DiHDPA, 16(17)-DiHDPA and 10(11)-DiHDPA, which are synthesized by hydroxylation of epoxygenated oxylipins in the CYP450 pathway (Table 5). As a result, product to substrate ratio of hydroxylated epoxides to epoxide 19(20)-DiHDPA/19(20)-EpDPA decreased, indicating suppressed degradation of epoxides with increased number of obstructed arteries as observed during hypoxia.

Six individual oxylipins were able to significantly distinguish between patients with 3 versus 1 or 2 obstructed arteries, all of which were lower in CAD3 participants: ARA-derived leukotriene B4 (ROC: 0.69; 95% CI: 0.57 to 0.81; P=0.003), EPA-derived 8-iso PGF3a (ROC: 0.67; 95% CI: 0.54 to 0.80; P=0.009), three DHA-derived DiHDPA 19(20)-DiHDPA (ROC: 0.66; 95% CI: 0.54 to 0.78; P=0.01), 16(17)-DiHDPA (ROC: 0.65; 95% CI: 0.52 to 0.79; P=0.02), 10(11)-DiHDPA (ROC: 0.64; 95% CI: 0.51 to 0.78; P=0.04), and LA-derived 12(13)-DiHOME (ROC: 0.64; 95% CI: 0.51 to 0.77; P=0.04). Using a cut-off value of leukotriene B4<0.21 nM for CAD3, leukotriene B4 classified CAD3 with 80% sensitivity (16 of 20 CAD3 participants) and 63% specificity (34 of 68 CAD1 and 2 participants; FIG. 3A). Each oxylipin alone had unsatisfactory classification ability; however, when combining leukotriene B4, 19(20)-EpDPA, 19(20)-DiHDPA, 13(14)-DiHDPA, and 10(11)-DiHDPA, an ROC-value of 0.90 (95% CI: 0.84 to 0.97; P<0.0001) was achieved, which could classify CAD3 with 100% sensitivity (20 of 20 CAD3 participants) and 68% specificity (46 of 68 CAD1 and CAD2 participants; FIG. 3B).

The concentration drop was more notable between CAD2 and CAD3 than between CAD1 and CAD2 patients. The only oxylipin, which could significantly distinguish between CAD patients with 1 versus more than 1 obstructed arteries was 8-iso PGF3a with an AUC ROC-value of 0.63 (95% CI: 0.50 to 0.76; P=0.04).

While illustrative embodiments have been illustrated and described, it will be appreciated that various changes can be made therein without departing from the spirit and scope of the invention.

### REFERENCES

1. Roth et al., Global, Regional, and National Burden of Cardiovascular Diseases for 10 Causes, 1990 to 2015. J. Am. Coll. Cardiol. 2017; 70(1):1-25.
2. Pagidipati and Gaziano, Estimating deaths from cardiovascular disease: a review of global methodologies of mortality measurement. Circulation. 2013; 127(6):749-756; doi:10.1161/CIRCULATIONAHA.112.128413
3. Nayeem M A. Role of oxylipins in cardiovascular diseases. Acta Pharmacol. Sin. 2018; 39(7):1142-1154.
4. WHO. The global burden of disease: 2004 update. Available at the WHO website.
5. Mathers and Loncar, Projections of global mortality and burden of disease from 2002 to 2030. PLoS Med. 2006 November; 3:e442.
6. Mozzafarian et al., on behalf of the American Heart Association Statistics Committee and Stroke Statistics Committee. Heart disease and stroke statistics—2016 update: a report from the American Heart Association. Circulation 2016; 133:e38-e360.
7. Hajar R. Risk Factors for Coronary Artery Disease: Historical Perspectives. Heart Views. 2017; 18(3):109-114.
8. Ambrose and Singh, Pathophysiology of coronary artery disease leading to acute coronary syndromes. F1000Prime Rep. 2015; 7:8.
9. Bentzon J et al., Mechanisms of plaque formation and rupture. Circ Res. 2014; 114(12):1852-1866. doi:10.1161/CIRCRESAHA.114.302721
10. Rafieian-Kopaei et al., Atherosclerosis: process, indicators, risk factors and new hopes. Int. J. Prev. Med. 2014; 5(8):927-946.
11. Zhong and Yin, Role of lipid peroxidation derived 4-hydroxynonenal (4-HNE) in cancer: focusing on mitochondria. Redox Biol. 2014; 4:193-199.
12. Gawel et al., [Malondialdehyde (MDA) as a lipid peroxidation marker]. Wiad Lek. 2004; 57(9-10):453-455.
13. Parthasarathy et al., Oxidized low-density lipoprotein. Methods Mol. Biol. 2010; 610:403-417.
14. Catala, Lipid peroxidation of membrane phospholipids generates hydroxy-alkenals and oxidized phospholipids active in physiological and/or pathological conditions. Chem. Phys. Lipids. 2009; 157(1):1-11.
15. Tourdot et al., The emerging role of oxylipins in thrombosis and diabetes. Front. Pharmacol. 2014; 4:176.
16. Kuller et al., Relation of C-reactive protein and coronary heart disease in the MRFIT nested case-control study. Multiple Risk Factor Intervention Trial. Am. J. Epidemiol. 1996; 144:537-47.
17. Ridker et al., C-reactive protein and coronary heart disease. N. Engl. J. Med. 2004; 351:295-298.
18. Johnson et al., Serum amyloid A as a predictor of coronary artery disease and cardiovascular outcome in women: The National Heart, Lung, and Blood Institute-Sponsored Women's Ischemia Syndrome Evaluation (WISE). Circulation 2004; 109:726-732.
19. Harb et al. Association of C-reactive protein and serum amyloid A with recurrent coronary events in stable patients after healing of acute myocardial infarction. Am. J. Cardiol. 2002; 89:216-221.
20. Liu et al., Elevated serum myeloperoxidase activities are significantly associated with the prevalence of ACS and High LDL-C levels in CHD patients. J. Atheroscler. Thromb. 2012; 19; 435-443.
21. Gabbs et al. Advances in our understanding of oxylipins derived from dietary PUFAs. Adv. Nutr. 2015; 6:513-540.
22. Mallat et al., The relationship of hydroxyeicosatetraenoic acids and F2-isoprostanes to plaque instability in human carotid atherosclerosis. J. Clin. Invest. 1999; 103:421-427.
23. Lundqvist et al., The arachidonate 15-lipoxygenase enzyme product 15-HETE is present in heart tissue from patients with ischemic heart disease and enhances clot formation. PLoS One. 2016; 11:1-13.
24. Strassburg et al., Quantitative profiling of oxylipins through comprehensive LC-MS/MS analysis: Application in cardiac surgery. Anal. Bioanal. Chem. 2012; 404:1413-1426.
25. Zu et al., Relationship between metabolites of arachidonic acid and prognosis in patients with acute coronary syndrome. Thromb. Res. 2016; 144:192-201.
26. Caligiuri et al., Specific plasma oxylipins increase the odds of cardiovascular and cerebrovascular events in patients with peripheral artery disease. Can. J. Physiol. Pharmacol. 2017; 95:961-968.
27. Shishehbor et al., Systemic elevations of free radical oxidation products of arachidonic acid are associated with angiographic evidence of coronary artery disease. Free Radic. Biol. Med. 2006; 41:1678-1683.
28. Xu et al., Exploratory investigation reveals parallel alteration of plasma fatty acids and eicosanoids in coronary artery disease patients. Prostaglandins Other Lipid Mediat. 2013; 106:29-36.
29. Auguet et al., Targeted metabolomic approach in men with carotid plaque. PLoS One. 2018; 13:1-11.
30. Pedersen and Newman, Establishing and Performing Targeted Multi-residue Analysis for Lipid Mediators and Fatty Acids in Small Clinical Plasma Samples. Methods Mol. Biol. 2018; 1730:175-212.
31. La Frano et al., Diet-induced obesity and weight loss alter bile acid concentrations and bile acid—sensitive gene expression in insulin target tissues of C57BL/6J mice. Nutr. Res. 2017; 46:11-21.
32. Morisseau and Hammock, Impact of Soluble Epoxide Hydrolase and Epoxyeicosanoids on Human Health. Annu. Rev. Pharmacol. Toxicol. 2012; 53(1):37-58.
33. Wagner et al., Soluble epoxide hydrolase as a therapeutic target for pain, inflammatory and neurodegenerative diseases. Pharmacol. Ther. 2017; 180:62-76.
34. Imig, Prospective for cytochrome P450 epoxygenase cardiovascular and renal therapeutics. Pharmacol. Ther. 2018; 192:1-19.
35. Fleming, The Pharmacology of the Cytochrome P450 Epoxygenase/Soluble Epoxide Hydrolase Axis in the Vasculature and Cardiovascular Disease. Pharmacol. Rev. 2014; 66(4): 1106-1140.
36. Dobrian et al., Functional and pathological roles of the 12- and 15-lipoxygenases. Prog. Lipid Res. 2011; 50(1):115-131.
37. Maayah and El-Kadi, The role of mid-chain hydroxyeicosatetraenoic acids in the pathogenesis of hypertension and cardiac hypertrophy. Arch. Toxicol. 2016; 90(1):119-136.
38. Garcia-Jaramillo et al., Lipidomic and transcriptomic analysis of western diet-induced nonalcoholic steatohepatitis (NASH) in female Ldlr−/− mice. PLoS One. 2019; 14(4):e0214387.
39. Garcia-Jaramillo et al., A Lipidomic Analysis of Docosahexaenoic Acid (22:6, ω3) Mediated Attenuation of Western Diet Induced Nonalcoholic Steatohepatitis in Male Ldlr−/− Mice. Metabolites 2019; 9(11):252.
40. Housley et al., Untargeted Metabolomic Screen Reveals Changes in Human Plasma Metabolite Profiles Following Consumption of Fresh Broccoli Sprouts. Mol. Nutr. Food Res. 2018; 62(19):e1700665.

1. An in vitro method for identifying a modified concentration (level) of at least one oxylipin in a biofluid sample obtained from a subject with a risk of coronary artery disease (CAD), the method comprising:
a. obtaining a biofluid sample from the subject;
b. detecting the concentration (level) of at least two oxylipins, and
c. comparing the concentration or level of the at least two oxylipins in the biofluid sample from the subject with a risk of CAD to a control level of the at least two oxylipins in at least one reference standard; wherein the concentration difference for each of the at least two oxylipins is decreased with the increase in the number of disease arteries, or at least two are decreased and one is increased wherein the subject has a higher chance of survival.

2. The method according to claim 1, wherein the at least two oxylipins are oxygenated omega-6 PUFA LA and ARA.

3. The method according to claim 2, wherein the at least two oxylipins are LA-derived mid-chain HODE and/or ARA-derived mid-chain HETE.

4. The method according to claim 1, wherein the at least two oxylipins comprise Leukotriene B4, 9(S)-HODE, 13(S)-HODE, 16(17)-DiHDPA, 13(14)-DiHDPA, 19(20)-EPDPA, 19(20)-DiHDPA, 10(11)-DiHDPA, 10(11)-EpDPA, or 7(8)-DiHDPA, or combinations thereof.

5. The method according to claim 4, wherein the at least two oxylipins comprise Leukotriene B4, 19(20)-DiHDPA, 13(14)-DiHDPA, and DiHDPA.

6. The method according to claim 4, wherein the at least two oxylipins comprise 9(S)-HODE, 16(17)-DiHDPA, 19(20)-EPDPA, 19(20)-DiHDPA, and 7(8)-DiHDPA.

7. The method according to claim 4, wherein the at least two oxylipins comprise 13(S)-HODE, and 10(11)-EpDPA.

8. The method according to claim 1, wherein the biofluid sample is a blood sample, a serum sample, a plasma sample, a urine sample, or a cerebrospinal fluid sample.

9. A method for treating CAD in a subject, said method comprising:
a. obtaining the results of an in vitro method, wherein said method comprises:
i. obtaining a biofluid sample from the subject;
ii. detecting the concentration (level) of at least two oxylipins; and
iii. comparing the concentration (level) of the at least two oxylipins in the biofluid sample from the subject with a risk of CAD to a control level of the at least two oxylipins in at least one reference standard from a subject not at risk of CAD; wherein the concentration difference for each of the at least two oxylipins is decreased with the increase in the number of disease arteries, and
b. treating the subject with coronary stent placement, or coronary artery bypass graft (CABG) surgery.

10. The method according to claim 1, wherein the at least two oxylipins are oxygenated omega-6 PUFA LA and ARA.

11. The method according to claim 10, wherein the at least two oxylipins are LA-derived mid-chain HODE and/or ARA-derived mid-chain HETE.

12. The method according to claim 9, wherein the at least two oxylipins comprise Leukotriene B4, 9(S)-HODE, 13(S)-HODE, 16(17)-DiHDPA, 13(14)-DiHDPA, 19(20)-EPDPA, 19(20)-DiHDPA, 10(11)-DiHDPA, 10(11)-EpDPA, or 7(8)-DiHDPA, or combinations thereof.

13. The method according to claim 12, wherein the at least two oxylipins comprise Leukotriene B4, 19(20)-DiHDPA, 13(14)-DiHDPA, and DiHDPA.

14. The method according to claim 12, wherein the at least two oxylipins comprise 9(S)-HODE, 16(17)-DiHDPA, 19(20)-EPDPA, 19(20)-DiHDPA, and 7(8)-DiHDPA.

15. The method according to claim 12, wherein the at least two oxylipins comprise 13(S)-HODE, and 10(11)-EpDPA.

16. The method according to claim 9, wherein the biofluid sample is a blood sample, a serum sample, a plasma sample, a urine sample, or cerebrospinal fluid.

17. A method for predicting survival of a subject at high risk of CAD comprising detecting a threshold amount of a LA-derived oxylipin, an EPA-derived oxylipin, an ARA-derived oxylipin, or combinations thereof.

18. The method according to claim 17, wherein the LA-derived oxylipin is one or more of 13(S)-HODE, 10(11)-EpDPA, 9(S)-HODE, 5-HETE, 8-iso PGF3α, and thromboxane B2.

19. The method according to claim 17, wherein the oxylipin is a combination of 13(S)-HODE and 10(11)-EpDPA, or 9(S)-HODE and 10(11)-EpDPA.

20. The method according to claim 17, wherein the subject does not require CABG and the oxylipin is 9(S)-HODE.
